(19) United States
(12) Patent Application Publication
(10) Pub. No.: US 2026/0193158 A1
(43) Pub. Date: Jul. 9, 2026

(54) IMPROVED PROCESS FOR PRODUCING ALKALI METAL METHOXIDES

(71) Applicant: Evonik Operations GmbH, Essen (DE)

(72) Inventors: Niklas PAUL, Marl (DE); Moritz SCHRÖDER, Münster (DE); Armin Matthias RIX, Mart (DE); Marius TIESBOHNENKAMP, Münster (DE); Dirk ROETTGER, Köln (DE); Tanita Valèrie SIX, Dortmund (DE); Patrick FRANCOIS, Essen (DE); Philp ZITZEWITZ, Haltern am See (DE); Min-Zae OH, Singapore (SG); Johannes RUWWE, Hanau (DE)

(73) Assignee: Evonik Operations GmbH, Essen (DE)

(21) Appl. No.: 19/132,848

(22) PCT Filed: Nov. 30, 2022
(86) PCT No.: PCT/EP2022/083797

§ 371 Date: May 26, 2025

(51) Int. Cl. C07C 29/70

(52) U.S. Cl. CPC C07C 29/705

(57) ABSTRACT

The present invention relates to a process for preparing at least one alkali metal methoxide by reactive distillation in at least one reaction column. At the lower end of the reaction column(s), the respective alkali metal methoxide dissolved in methanol is withdrawn. The methanol/water mixture obtained at the top of the reaction column(s) is separated by distillation in a rectification column.

The vapours obtained at the upper end of the rectification column are compressed in at least two stages and the energy of the vapours compressed in each case is advantageously transferred to bottoms and side streams of the rectification column. This allows particularly efficient use of the energy of the compressed vapours in the process according to the invention. In addition, the energy in the product stream of alkali metal methoxide which is obtained at the lower end of the reaction column(s) is advantageously transferred to the vapour streams from the rectification column, especially before these vapours are subjected to compression.

The energy from the compressed vapour may additionally be used for operation of the reaction column(s) or for operation of a reaction column in which a process for transalcoholization of alkali metal alkoxides is performed.

The present invention relates to a process for preparing at least one alkali metal methoxide by reactive distillation in at least one reaction column. At the lower end of the reaction column(s), the respective alkali metal methoxide dissolved in methanol is withdrawn. The methanol/water mixture obtained at the top of the reaction column(s) is separated by distillation in a rectification column.

The vapours obtained at the upper end of the rectification column are compressed in at least two stages and the energy of the vapours compressed in each case is advantageously transferred to bottoms and side streams of the rectification column. This allows particularly efficient use of the energy of the compressed vapours in the process according to the invention. In addition, the energy in the product stream of alkali metal methoxide which is obtained at the lower end of the reaction column(s) is advantageously transferred to the vapour streams from the rectification column, especially before these vapours are subjected to compression.

The energy from the compressed vapour may additionally be used for operation of the reaction column(s) or for operation of a reaction column in which a process for transalcoholization of alkali metal alkoxides is performed.

### 1. BACKGROUND OF THE INVENTION

Alkali metal alkoxides are used as strong bases in the synthesis of numerous chemicals, for example in the production of pharmaceutical or agrochemical active ingredients. Alkali metal alkoxides are also used as catalysts in transesterification and amidation reactions.

Alkali metal alkoxides are prepared by electrolysis, for example, as described in EP 3 885 470 A1.

A further means of preparing alkali metal alkoxides is the reaction of an alkali metal alkoxide with another alcohol. This “transalcoholization” is described, for example, in CS 213119 B1. It is advantageously conducted in a reaction column, as described in WO 2021/122702 A1, U.S. Pat. No. 3,418,383 A and DE 27 26 491 A1.

Alternatively, and most commonly, alkali metal alkoxides (MOR) are prepared by reactive distillation of alkali metal hydroxides (MOH) and alcohols (methanol) in a countercurrent distillation column, wherein the water of reaction formed according to the following reaction <1> is removed with the distillate.

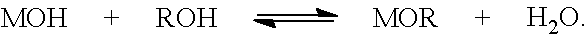

Such a process principle is described, for example, in U.S. Pat. No. 2,877,274 A, WO 01/42178 A1, CN 109 627 145 A, CN 208632416 U, WO 2021/148174 A1 and WO 2021/148175 A1, wherein aqueous alkali metal hydroxide solution and gaseous methanol are run in countercurrent in at least one reactive rectification column.

Methods that are similar, but in which an entraining agent, for example benzene, is additionally used, are described in GB 377,631 A and U.S. Pat. No. 1,910,331 A. This entraining agent is used to separate water and the water-soluble alcohol. In both patent specifications the condensate is subjected to a phase separation to separate off the water of reaction.

Correspondingly, DE 96 89 03 C describes a method of continuous preparation of alkali metal alkoxides in a reaction column, wherein the water-alcohol mixture withdrawn at the top of the column is condensed and then subjected to a phase separation. The aqueous phase is discarded and the alcoholic phase is returned to the top of the column together with the fresh alcohol. EP 0 299 577 A2 describes a similar method, wherein the water in the condensate is separated off with the aid of a membrane.

The most industrially important alkali metal alkoxides are those of sodium and potassium, especially the methoxides and ethoxides. Their synthesis is frequently described in the prior art, for example in EP 1 997 794 A1.

The syntheses of alkali metal alkoxides by reactive rectification described in the prior art typically afford vapours comprising the alcohol used and water. It is advantageous for economic reasons to reuse the alcohol present in the vapours as a reactant in the reactive distillation. The vapours are therefore typically supplied to a rectification column and the alcohol present therein is separated off (described for example in GB 737 453 A and U.S. Pat. No. 4,566,947 A). The alcohol thus recovered is then fed to the reactive distillation as a reactant for example.

Alternatively or additionally, a portion of the alcohol vapour may be utilized for heating the rectification column, as described, for example, in WO 2010/097318 A1, EP 4 074 684 A1 and EP 4 074 685 A1. However, this requires that the vapour be compressed in order to achieve the temperature level required for heating the rectification column. In particular, a multistage compression of the vapour is thermodynamically advantageous. The vapour is cooled here between the compression stages. The intermediate cooling also ensures that the maximum permissible temperature of the compressor is not exceeded. The disadvantage of this cooling performed in the conventional processes is that the energy thus withdrawn is dissipated without being utilized.

There is accordingly the need for an improved process for preparing alkali metal methoxides in which the obtained methanol/water mixture is worked up with a rectification column. This process is to feature a low total energy requirement and, in particular, particularly efficient utilization of the energy present in the compressed vapour for operation of the rectification column. The process is thus to enable efficient utilization of the heat obtained in the compression and cooling of the vapours. In addition, the method is to be particularly gentle in respect of the apparatuses (compressors) used in the compression and hence to prolong the lifetime thereof. Finally, the process is to simplify the handling of the product streams obtained from the reaction columns, for instance in processing and storage.

### 2. Brief Summary of the Invention

The present invention solves the problem according to the invention and relates to a process for preparing at least one alkali metal methoxide of the formula M_{A}OCH₃, where M_{A} is selected from sodium, potassium, lithium, especially selected from sodium, potassium, and is preferably sodium.

Optionally, simultaneously and spatially separately from the conversion to the alkali metal methoxide of the formula M_{A}OCH₃, in a second reaction column RR_{B}, a further alkali metal methoxide of the formula M_{B}OCH₃ is prepared, where M_{B} is selected from sodium, potassium, lithium, especially from sodium, potassium, and is preferably potassium.

This affords, at the top of the reaction column RR_{A} or of the reaction columns RR_{A} and RR_{B}, one vapour stream S_{AB} or two vapour streams S_{AB} and S_{BB}, each comprising water and methanol. Stream S_{AB} or streams S_{AB} and S_{BB} are directed separately from one another (i.e. not mixed with one another) or mixed with one another into a rectification column RD_{A}, where they are separated by distillation into water and methanol. Methanol is obtained at the top of RD_{A} as vapour stream S_{OA}. The vapour stream S_{OA} is compressed in at least two stages to give S_{OA1} (stage 1) and S_{OA2} (stage 2), and the energy from the respectively compressed vapour stream S_{OA1} or S_{OA2} or a portion thereof is advantageously integrated into the process in that it is transferred to a sidestream S_{ZA} withdrawn from the rectification column RD_{A} (in the case of S_{OA1}), or bottom stream S_{UA} or a portion S_{UA1} thereof (in the case of S_{OA2}).

In addition, energy is transferred from the bottom product stream S_{AP} from the rectification column RR_{A} and/or from the bottom product stream SBP from the reaction column RR_{B} to at least a portion of at least one of the streams S_{OA}, S_{OA1}, S_{OA2}, which even further improves thermal integration and hence energy efficiency.

This transfer of energy from the bottom product stream S_{AP} and/or from the bottom product stream S_{BP} to at least a portion of at least one of streams S_{OA}, S_{OA1}, S_{OA2} preferably precedes compression of the stream in question S_{OA}, S_{OA1}, S_{OA2} to which energy is transferred. This prevents the forming of a liquid phase that could damage the compressor apparatus in the stream to be compressed before and/or during the compression.

A further advantageous effect is that, at the same time, the bottom product stream S_{AP} or the bottom product stream S_{BP} is cooled without dissipation of the energy unutilized and without any need to expend additional energy for cooling. The bottom product stream S_{AP} or the bottom product stream S_{BP} is thus easier to handle in the course of processing or storage.

In a further preferred aspect, the present invention relates to a process for transalcoholization of alkali metal alkoxides, which is especially conducted in a reaction column. In this process, the alcohol radical of an alkali metal alkoxide M_{c}OR′ is exchanged for another alcohol R″OH, where R′ and R″ are two different C₁ to C₇ hydrocarbon radicals; in particular, R′=methyl and R″=C₂ to C₇ hydrocarbon radical, preferably R′=methyl and R″=ethyl, n-propyl, iso-propyl, more preferably R′=methyl and R″=ethyl. This is done using energy from at least a portion of a stream selected from S_{OA1}, S_{OA2} in the process for transalcoholization.

### 3. FIGS

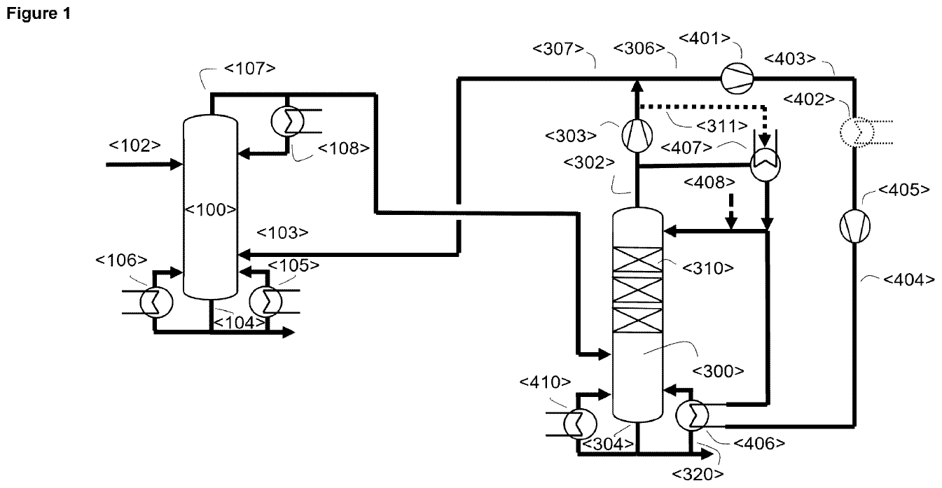

### 3.1 FIG. 1

FIG. 1 shows a noninventive embodiment of a process for preparing alkali metal methoxides, in which the methanol-water mixture is separated by distillation as in the prior art (corresponding essentially to WO 2010/097318 A1, FIG. 1).

This involves reacting aqueous NaOH solution in a reaction column RR_{A} <100> with methanol to give sodium methoxide. At the top of the reaction column RR_{A} <100> an aqueous NaOH solution is added as reactant stream S_{AE2} <102>. It is alternatively also possible to add a methanolic NaOH solution as reactant stream S_{AE2} <102>. The corresponding potassium methoxide is prepared by adding aqueous or methanolic KOH solution as reactant stream S_{AE2} <102>. The corresponding lithium methoxide is prepared by adding aqueous or methanolic LiOH solution as reactant stream S_{AE2} <102>. Above the bottom of the reaction column RR_{A} <100>, methanol is added in vapour form as reactant stream S_{AE1} <103>.

At the bottom of the reaction column RR_{A} <100>, a mixture of the corresponding alkali metal methoxide in methanol S_{AP} <104> is withdrawn. The reboiler V_{SA} <105> and the optional evaporator V_{SA′} <106> at the bottom of the column RR_{A} <100> are used to adjust the concentration of the sodium methoxide solution (or KOCH₃ or LiOCH₃ solution) obtained as S_{AP} <104> to the desired value.

At the top of the reaction column RR_{A} <100>, a vapour stream S_{AB} <107> is withdrawn. A portion of the vapour stream S_{AB} <107> is condensed in the condenser K_{RRA} <108> and applied in liquid form to the top of the reaction column RR_{A} <100> as reflux. However, the condenser K_{RRA} <108> and the establishment of the reflux are optional. The adjustment of the concentration of the sodium methoxide solution S_{AP} <104> to the desired value can also be controlled via the reflux.

The resultant vapour stream S_{AB} <107> is fed wholly or partly to a rectification column RD_{A} <300>, which is a column for separation of water and methanol. The rectification column RD_{A} <300> contains internals <310>. The vapour stream S_{AB} <107> is separated by distillation therein, and methanol is recovered by distillation overhead as vapour stream S_{OA} <302>. A reflux may be established in the rectification column RD_{A} <300>. In that case, a portion of the vapour stream S_{OA} <302> is condensed in a condenser K_{RD} <407> and then recycled back into the rectification column RD_{A} <300>. In the embodiments where no reflux is established, the remaining portion of S_{OA} <302>, or the complete vapour stream S_{OA} <302>, is precompressed using compressor VD_{AB2} <303>.

If a reflux is established, the portion of S_{OA} <302> intended as reflux may alternatively or additionally, especially alternatively, also first be separated from S_{OA} <302> after S_{OA} <302> has been precompressed in the compressor VD_{AB2} <303>. This option is reflected in the respective figures by the dotted line <311>.

A portion [symbol] <307> of this precompressed vapour S_{OA} <302> is recycled to the reaction column RR_{A} <100>, where it is used as reactant stream S_{AE1} <103>.

The remaining portion [symbol] <306> of S_{OA} <302> is fed to the compressor VD₁ <401>, where it is further compressed to give vapour stream S_{OA1} <403>, from which energy can be removed in the optional intermediate cooler WTₓ <402> (illustrated by dotted lines in FIG. 1 and the respective figures). The vapour stream S_{OA1} <403> is compressed again using compressor VDₓ <405> and the resulting vapour S_{OA2} <404> is fed to the evaporator V_{SRD} <406> at the bottom of the rectification column RD_{A} <300> for heating, and then a stream of fresh methanol <408> (shown by a dotted line) is optionally added thereto and it is fed back to the rectification column RD_{A} <300>. If a reflux is established in the rectification column RD_{A} <300>, the stream S_{OA2} <404>, before being returned to RD_{A} <300>, may be mixed with the reflux, i.e. the condensate, from K_{RD} <407> and fed into RD_{A} <300> together therewith. Obtained at the bottom of the rectification column RD_{A} <300> is a water stream S_{UA} <304> which is at least partly (stream S_{UA1} <320>) recycled back into the rectification column RD_{A} <300>, in which case it is directed through the reboiler V_{SRD} <406>. Another portion can be directed via the reboiler V_{SRD′} <410>.

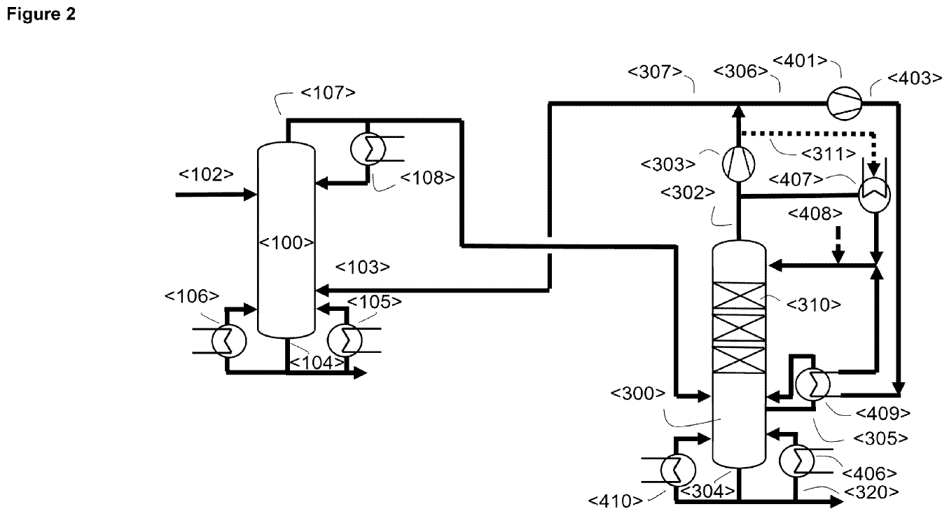

### 3.2 FIG. 2

FIG. 2 shows a further noninventive embodiment of a process which corresponds essentially to the embodiment shown in FIG. 2 of WO 2010/097318 A1.

This corresponds to the embodiment described in FIG. 1 with the following additional/different features: In addition to the evaporators V_{SRD′} <406> and V_{SRD′} <410> at the bottom, the rectification column RD_{A} <300> has an intermediate evaporator V_{ZRD} <409>. A sidestream S_{ZA} <305> is withdrawn from the rectification column RD_{A} <300> and directed through the intermediate evaporator V_{ZRD} <409>, then fed back to the rectification column RD_{A} <300>. A portion of the vapour stream S_{OA} <302> is precompressed by means of compressor VD_{AB2} <303>. The reflux is branched off from the vapour stream S_{OA} <302> before the vapour stream S_{OA} <302> passes through the precompressor VD_{AB2} <303>. Alternatively or additionally, the reflux <311> may also be branched off from S_{OA} <302> after it has passed through the precompressor VD_{AB2} <303>. Once the substream [symbol] <307> to the reaction column RR_{A} <100> has been branched off, the remaining substream [symbol] <306> is compressed in the compressor VD₁ <401> to give the vapour stream S_{OA1} <403>, and this is fed to the intermediate evaporator V_{ZRD} <409> for heating. There is no heating in evaporator V_{SRD} <406> or in evaporator V_{SRD′} <410> by S_{OA1} <403>. The vapour stream S_{OA1} <403> utilized for heating of V_{ZRD} <409> is then mixed with the condensate from K_{RD} <407> and optionally a stream of fresh methanol <408> and returned to the rectification column RD_{A} <300> as reflux. Separate recycling of the reflux from S_{OA} <302> to the rectification column RD_{A} <300> is likewise possible.

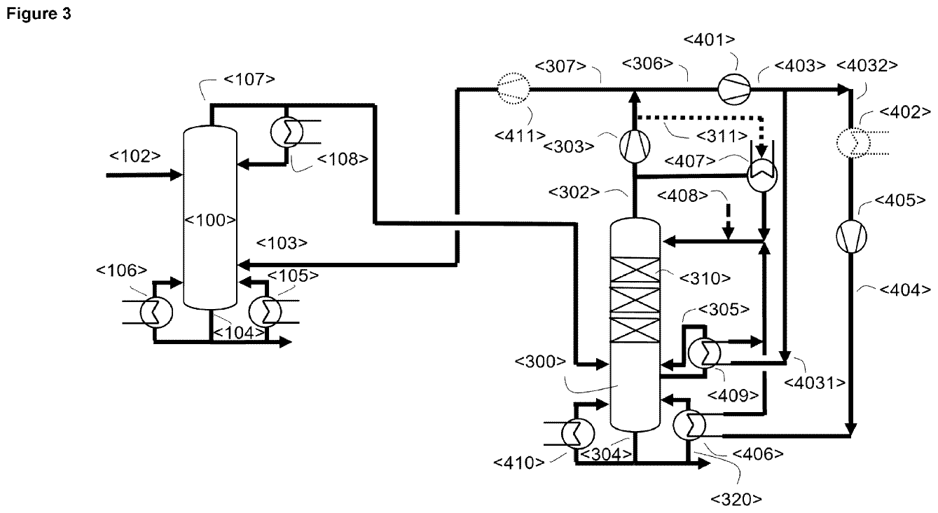

### 3.3 FIG. 3

FIG. 3 shows a further embodiment of a noninventive process. This corresponds to the embodiments described in FIGS. 1 and 2. The rectification column RD_{A} <300> has an intermediate evaporator V_{ZRD} <409> and a reboiler V_{SRD} <406> and optionally the reboiler V_{SRD′} <410>.

The inventive embodiment has the following differences from the aforementioned embodiments:
1. After compression of the portion [symbol] <306> of the vapour stream S_{OA} <302> in the compressor VD₁ <401>, the vapour stream S_{OA1} <403> is divided into two portions S_{OA11} <4031> and S_{OA12} <4032>.
2. S_{OA1} <4031> is supplied to the intermediate evaporator V_{ZRD} <409> for heating the stream S_{ZA} <305>.
3. S_{OA12} <4032> is further compressed in the compressor VDₓ <405> to give stream S_{OA2} <404> and S_{OA2} <404> is fed to the reboiler V_{SRD} <406> for heating of stream S_{UA1} <320>.
4. Once S_{OA11} <4031> and S_{OA2} <404> have left the respective evaporator V_{ZRD} <409>/V_{SRD} <406>, they are combined and the combined stream is mixed with the reflux (i.e. the condensate from K_{RD} <407>) and optionally the stream of fresh methanol <408> and recycled to the rectification column RD_{A} <300>. For this, these streams may also first be collected in a condensate vessel as described in FIG. 11 (condensate vessel <419>). Separate recycling of these streams to the rectification column RD_{A} <300> is likewise possible.
5. After [symbol] <307> has been separated from S_{OA} <302>, it is optionally compressed by means of a compressor VD_{♥} <411> before being used as reactant stream S_{AE1} <103> in the reaction column RR_{A} <100>. This use of an additional compressor VD_{♥} <411> permits adjustment of the pressure of the stream [symbol] <307> reused as reactant stream S_{AE1} <103> exactly to the required pressure conditions in the reaction column RR_{A} <100>.

The differences from the embodiments according to FIGS. 1 and 2 are thus that the compression of the vapour stream S_{OA} is conducted in two stages, first to give the vapour S_{OA1} <403> and then to give the vapour S_{OA2} <404>. Because the less compressed vapour stream S_{OA1} <403> or the portion S_{OA11} <4031> thereof is used for the heating of the sidestream S_{ZA} <305>, while the more strongly compressed vapour is used for the heating of the portion S_{UA1} <320> of the bottom stream S_{UA} <304>, the rectification column RD_{A} <300> is heated more efficiently by the vapour stream by comparison with the embodiment according to FIGS. 1 and 2.

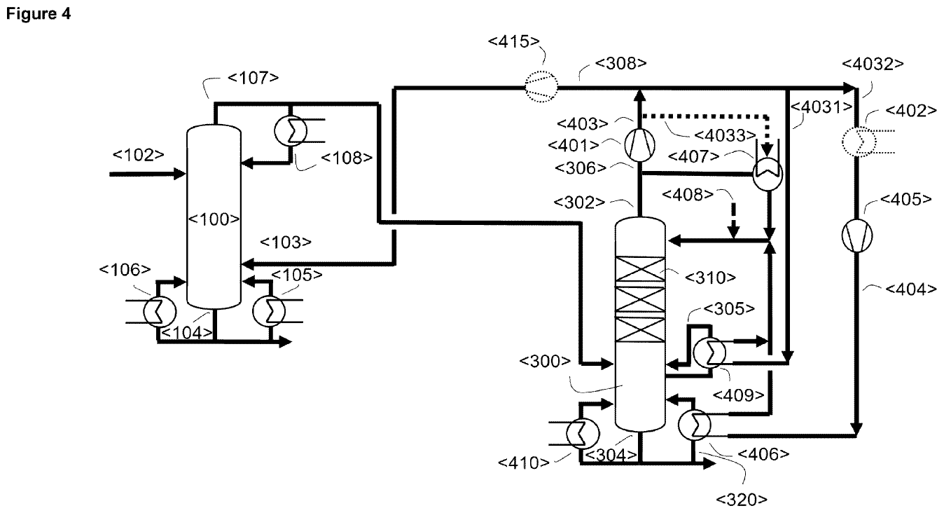

### 3.4 FIG. 4

FIG. 4 shows a further embodiment of a noninventive process.

This corresponds to the embodiment described in FIG. 3 with the following differences:
1. There is no precompression of the vapour stream S_{OA} by means of a compressor VD_{AB2} <303>. The portion [symbol] <306> of the vapour stream S_{OA} <302> which is fed to the compressor VD₁ <401> and compressed to give S_{OA1} <403> corresponds to the vapour stream S_{OA} <302> minus the portion which is condensed by means of the condenser K_{RD} <407> and then recycled back to the rectification column RD_{A} <300>.
2. In this embodiment, it is additionally or alternatively, preferably alternatively, possible also to condense a portion of the compressed vapour stream S_{OA1} <403> as reflux S_{OA13} <4033> via the condenser K_{RD} <407> and to recycle the condensate to the rectification column RD_{A} <300>.
3. A portion S_{OA♥} <308> is separated from S_{OA1} <403> and used as reactant stream S_{AE1} <103>. It is possible here to compress S_{OA♥} <308> by means of an optional compressor VD_{♥} <415>. S_{OA♥} <308>, as is self-evident, is different from S_{OA11} <4031> and from S_{OA12} <4032> and of course also from S_{OA13} <4033>.

As already described for the embodiment according to FIG. 3, in the embodiment according to FIG. 4 as well, an energy saving arises in that S_{OA1} <4031> is fed to the intermediate evaporator V_{ZRD} <409> for heating of stream S_{ZA} <305> and S_{OA12} <4032> is additionally compressed in compressor VDₓ <405> to give stream S_{OA2} <404>, and this compressed stream is then fed to the reboiler V_{SRD} <406> for heating of stream S_{UA1} <320>. This division of the differently compressed streams between sidestream S_{ZA} <305> and bottom stream S_{UA1} <320> results in a higher energy efficiency in the energy transfer.

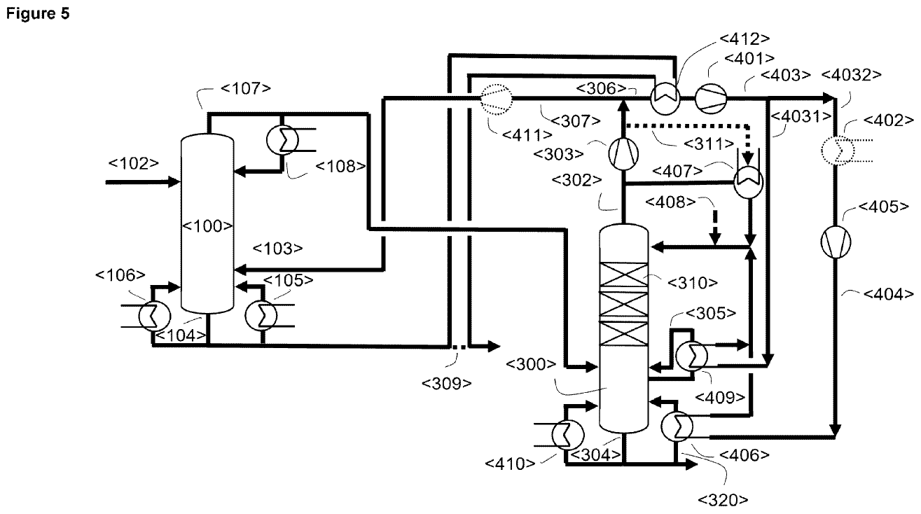

### 3.5 FIG. 5

FIG. 5 shows one embodiment of the process according to the invention. It corresponds to the embodiment described in FIG. 3, except that energy is transferred by means of a heat transferrer [symbol] <412> in the form of heat from S_{AP} <104> to [symbol] <306>. In addition to the energy saving that has been described for the embodiment according to FIGS. 3 and 4, energy is additionally saved in that the residual heat in the bottom stream S_{AP} <104> does not dissipate unutilized, but is reintegrated into the process. This additional energy integration is also advantageous in that the vapour stream [symbol] <306> is heated by means of heat exchanger [symbol] <412> before it is compressed in the compressor VD₁ <401>. This heating will convert any condensed droplets present in the vapour stream [symbol] <306> to the gaseous state, or prevent the unwanted condensation of the vapour before or during the compression. Such droplets can have an adverse effect on the mechanical structure of the compressor, and the avoidance thereof thus increases the life of the apparatus. It is also possible to utilize only the energy of a portion of stream S_{AP} <104>. In that case, only the energy of a portion of stream S_{AP} <104> is transferred, while the energy of another portion <309> of the stream is not utilized. The portion <309> is indicated by the dotted line.

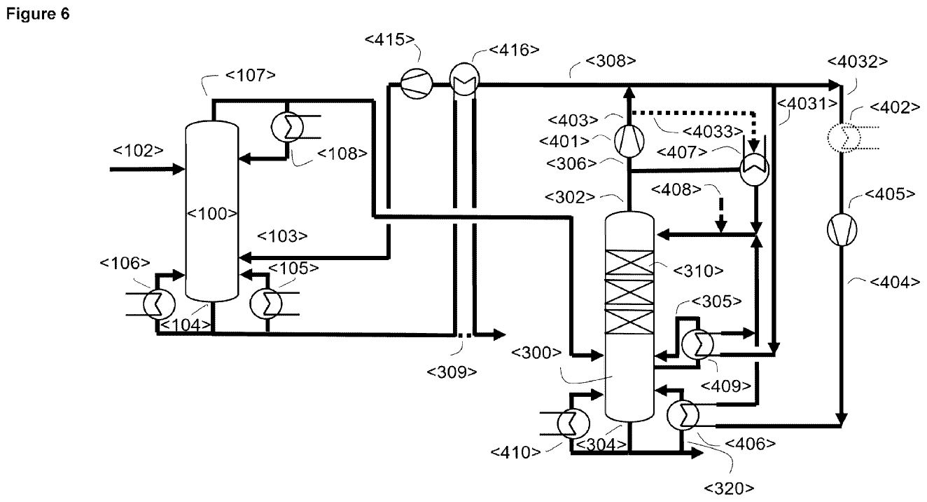

### 3.6 FIG. 6

FIG. 6 shows a further embodiment of the process according to the invention. It corresponds to the embodiment described in FIG. 4, but the compressor VD_{♥} <415> is compulsory rather than optional.

Further differences from the embodiment according to FIG. 4 are that energy is transferred by means of a heat transferrer WT_{♥} <416> in the form of heat from S_{AP} <104> to S_{OA♥} <308>. In addition to the advantages of energy saving that have been described for the embodiment according to FIGS. 3 and 4, energy is additionally thereby saved since the residual heat in the bottom stream S_{AP} <104> does not dissipate unutilized, but is reintegrated into the process. The integration of the energy from S_{AP} <104> into the vapour stream S_{OA♥} <308> via heat exchanger WT_{♥} <416> before S_{OA♥} <308> is compressed in the compressor VD_{♥} <415> brings additional advantages. The heating will convert any condensed droplets present in the vapour stream S_{OA♥} <308> to the gaseous state, or prevent the unwanted condensation of the vapour stream S_{OA♥} <308> before or during the compression. Such droplets can have an adverse effect on the mechanical structure of the compressor, and the avoidance thereof thus increases the life of the apparatus. It is also possible to utilize only the energy of a portion of stream S_{AP} <104>. In that case, only the energy of a portion of stream S_{AP} <104> is transferred, while the energy of another portion <309> of the stream is not utilized. The portion <309> is indicated by the dotted line.

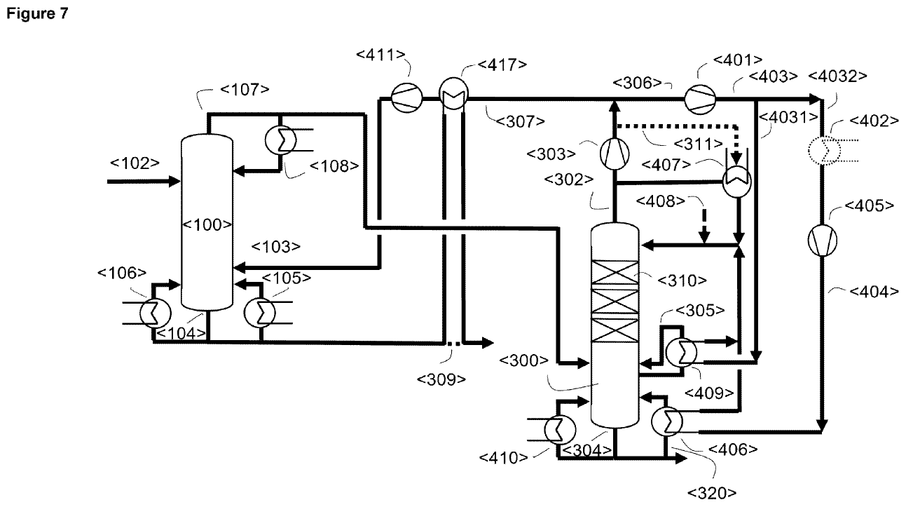

### 3.7 FIG. 7

FIG. 7 shows a further embodiment of the process according to the invention. It corresponds to the embodiment described in FIG. 3, but the compressor [symbol] <411> is compulsory rather than optional.

Further differences from embodiment 3 are that energy is transferred by means of a heat transferrer [symbol] <417> in the form of heat from S_{AP} <104> to [symbol] <307>. In addition to the advantages of energy saving that have been described for the embodiment according to FIG. 3, energy is additionally thereby saved since the residual heat in the bottom stream S_{AP} <104> does not dissipate unutilized, but is reintegrated into the process. The integration of the energy from S_{AP} <104> via heat transferrer [symbol] <417> is also additionally thereby advantageous since the vapour stream [symbol] <307> is heated before it is compressed in the compressor [symbol] <411>. The heating will convert any condensed droplets present in the vapour stream [symbol] <307> to the gaseous state, or prevent the unwanted condensation of the vapour before or during the compression. Such droplets can have an adverse effect on the mechanical structure of the compressor, and the avoidance thereof thus increases the life of the apparatus. It is also possible to utilize only the energy of a portion of stream S_{AP} <104>. In that case, only the energy of a portion of stream S_{AP} <104> is transferred, while the energy of another portion <309> of the stream is not utilized. The portion <309> is indicated by the dotted line.

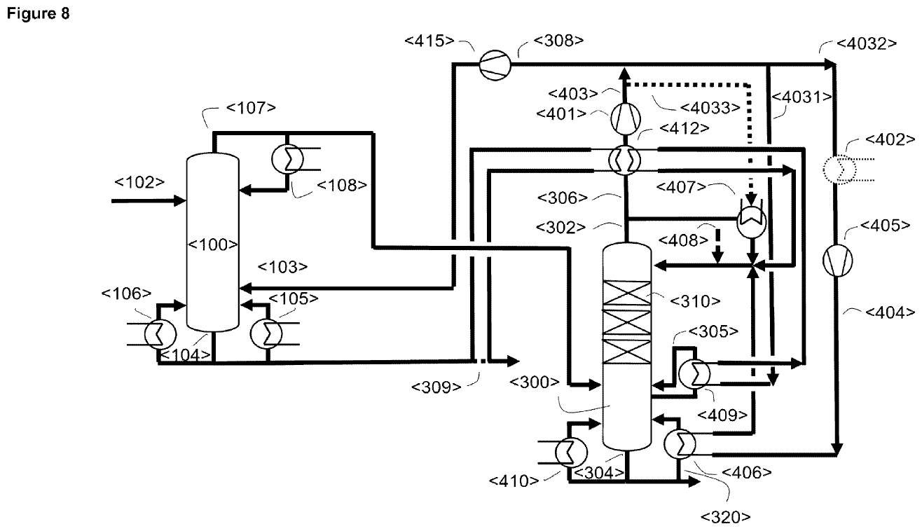

### 3.8 FIG. 8

FIG. 8 shows a further embodiment of the process according to the invention.

It corresponds to the embodiment described in FIG. 4, but the compressor VD_{♥} <415> is compulsory rather than optional.

Further differences from embodiment 4 are that energy is transferred by means of a heat transferrer [symbol] <412> in the form of heat from S_{AP} <104> to [symbol] <306>. In addition, stream S_{OA11} <4031>, after it has transferred energy in the intermediate evaporator V_{ZRD} <409> to the sidestream S_{ZA} <305>, is likewise directed through the heat transferrer [symbol] <412> in order to transfer the residual energy in the form of heat from S_{OA11} <4031> to [symbol] <306>.

In addition to the advantages of energy saving that have been described for the embodiment according to FIG. 4, energy is additionally thereby saved since the residual heat in the bottom stream S_{AP} <104> and in stream S_{OA11} <4031> does not dissipate unutilized, but is reintegrated into the process. The integration of the energy from S_{AP} <104> and S_{OA11} <4031> by means of heat exchanger [symbol] <412> is additionally advantageous in that it is effected on the vapour stream [symbol] <306> before the latter is compressed in the compressor VD₁ <401>. The heating will convert any condensed droplets present in the vapour stream [symbol] <306> to the gaseous state, or prevent the unwanted condensation of the vapour [symbol] <306> before or during the compression. Such droplets can have an adverse effect on the mechanical structure of the compressor, and the avoidance thereof thus increases the life of the apparatus. It is also possible to utilize only the energy of a portion of stream S_{AP} <104>. In that case, only the energy of a portion of stream S_{AP} <104> is transferred, while the energy of another portion <309> of the stream is not utilized. The portion <309> is indicated by the dotted line. It is likewise also possible to use only the residual heat from a portion of stream S_{OA11} <4031> for the energy integration via [symbol] <412> (not shown in FIG. 8).

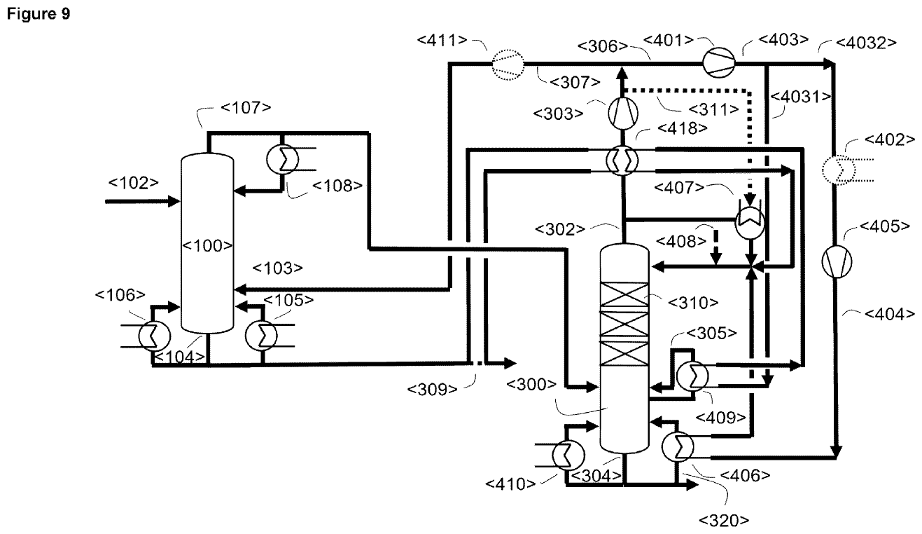

### 3.9 FIG. 9

FIG. 9 shows one embodiment of the process according to the invention. This corresponds to the embodiment described in FIG. 3.

Differences from embodiment 3 are that a heat transferrer WT_{AB2} <418> is used to transfer energy in the form of heat from S_{AP} <104> to a portion of the stream of S_{OA} <302>, which is precompressed in the compressor VD_{AB2} <303>.

In addition, stream S_{OA11} <4031>, after it has transferred energy in the intermediate evaporator V_{ZRD} <409> to the sidestream S_{ZA} <305>, is likewise directed through the heat transferrer WT_{AB2} <418> in order to transfer the residual energy in the form of heat from S_{OA11} <4031> to the portion of stream S_{OA} <302> that is to be precompressed by means of VD_{AB2} <303>.

In addition to the advantages of energy saving that have been described for the embodiment according to FIG. 3, energy is additionally thereby saved since the residual heat in the bottom stream S_{AP} <104> and in stream S_{OA11} <4031> does not dissipate unutilized, but is reintegrated into the process. The integration of the energy from S_{AP} <104> and S_{OA11} <4031> by means of the heat transferrer WT_{AB2} <418> is also additionally thereby advantageous since the portion of the vapour stream S_{OA} <302> to be precompressed is heated before it is precompressed in the compressor VD_{AB2} <303>. The heating will convert any condensed droplets present in this portion of the vapour stream S_{OA} <302> to the gaseous state, or prevent the unwanted condensation of the vapour S_{OA} <302> before or during the precompression. Such droplets can have an adverse effect on the mechanical structure of the compressor, and the avoidance thereof thus increases the life of the apparatus. It is also possible to utilize only the energy of a portion of stream S_{AP} <104>. In that case, only the energy of a portion of stream S_{AP} <104> is transferred, while the energy of another portion <309> of the stream is not utilized. The portion <309> is indicated by the dotted line. It is likewise also possible to use only the residual heat from a portion of stream S_{OA11} <4031> (not shown in FIG. 9).

The process conducted in Inventive Example 4 (section 5.4) was conducted using the apparatus shown in FIG. 9, without in Example 4 conducting the compression of stream [symbol] <307> with compressor [symbol] <411> which is shown as optional in FIG. 9.

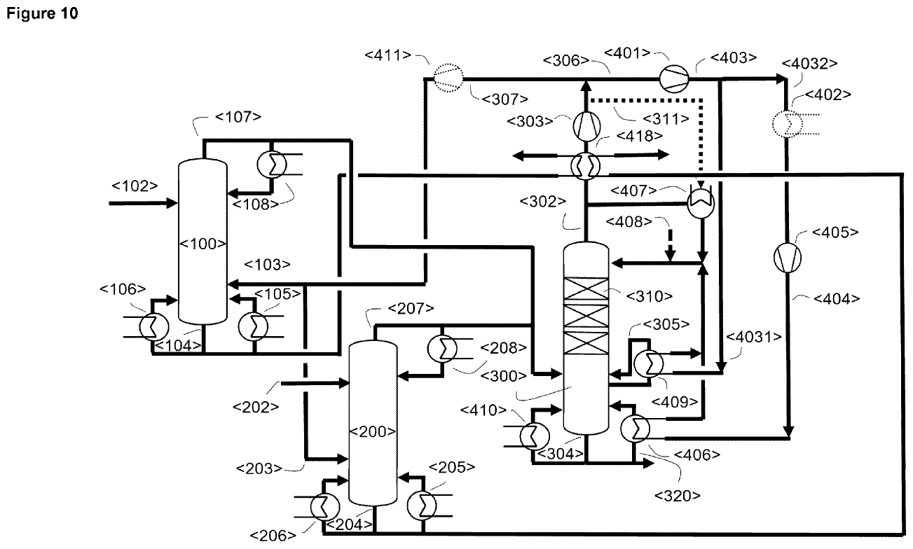

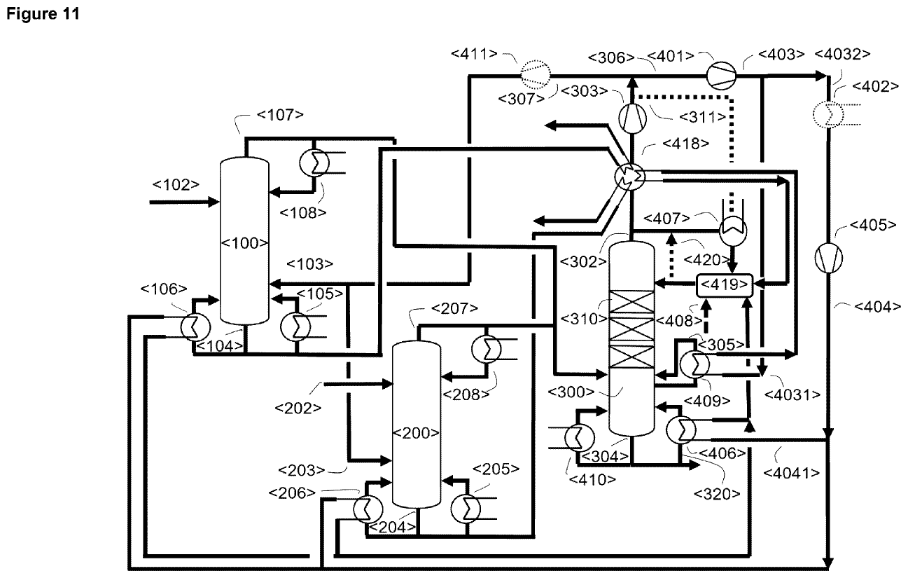

### 3.10 FIG. 10

FIG. 10 shows one embodiment of the process according to the invention. This corresponds to the embodiment described in FIG. 3 with the following differences:

In a second reaction column RR_{B} <200>, an aqueous KOH solution is reacted with methanol that is also used for reaction in RR_{A} <100> to give potassium methoxide.

At the top of the reaction column RR_{B} <200> an aqueous KOH solution is added as reactant stream S_{BE2} <202>. It is alternatively possible to add a methanolic KOH solution as reactant stream S_{BE2} <202>. Above the bottom of the reaction column RR_{B} <200>, methanol is added in vapour form as reactant stream S_{BE1} <203>.

At the bottom of the reaction column RR_{B} <200>, a mixture of the corresponding methoxide in methanol S_{BP} <204> is withdrawn. The reboiler V_{SB} <205> and the optional evaporator V_{SB′} <206> at the bottom of the column RR_{B} <200> are used to adjust the concentration of the potassium methoxide solution S_{BP} <204> to the desired value.

At the top of the reaction column RR_{B} <200>, a vapour stream S_{BB} <207> is withdrawn. A portion of the vapour stream S_{BB} <207> is condensed in the condenser K_{RRB} <208> and applied in liquid form as reflux to the top of the reaction column RR_{B} <200>. However, the condenser K_{RRB} <208> and the establishment of the reflux are optional. The adjustment of the concentration of the potassium methoxide solution S_{BP} <204> to the desired value can also be controlled via the reflux.

The obtained vapour S_{BB} <207> is supplied to the rectification column RD_{A} <300> in admixture with the portion of the vapour S_{AB} <107> not condensed in the condenser K_{RRA} <108>.

A further difference from the embodiment according to FIG. 3 is that the precompressed portion [symbol] <307> of the vapour stream S_{OA} <302> is recycled to the reaction column RR_{A} <100> and to the reaction column RR_{B} <200>, where it is used as reactant stream S_{AE1} <103>/S_{BE1} <203>. A further difference from the embodiment according to FIG. 3 is that a heat transferrer WT_{AB2} <418> is used to transfer energy in the form of heat from S_{AP} <104> to a portion of the stream of S_{OA} <302>, which is precompressed in the compressor V_{DAB2} <303>.

In addition, stream S_{BP} <204> is likewise directed through the heat transferrer WT_{AB2} <418> in order to transfer the residual energy in the form of heat from S_{BP} <204> to the portion of stream S_{OA} <302> that has been precompressed by means of V_{DAB2} <303>.

In addition to the advantages of energy saving that have been described for the embodiment according to FIG. 3, energy is additionally thereby saved since the residual heat in the two bottom streams S_{AP} <104> and S_{BP} <204> does not dissipate unutilized, but is reintegrated into the process. The integration of the energy in S_{AP} <104> and S_{BP} <204> by means of heat transferrer WT_{AB2} <418> is additionally advantageous in that the portion of the vapour stream S_{OA} <302> to be precompressed is heated before it is precompressed in the compressor VD_{AB2} <303>. The heating will convert any condensed droplets present in the vapour stream S_{OA} <302> to the gaseous state, or prevent the unwanted condensation of S_{OA} <302> before or during the precompression. Such droplets can have an adverse effect on the mechanical structure of the compressor, and the avoidance thereof thus increases the life of the apparatus. It is also possible to utilize only the energy of a portion of stream S_{AP} <104> and/or of a portion of stream S_{BP} <204> for integration of the energy thereof via WT_{AB2} <418> (not shown in FIG. 10).

### 3.11 FIG. 11

FIG. 11 shows one embodiment of the process according to the invention. It corresponds to the embodiments described in FIG. 10 with the following differences:
1) Only a portion S_{OA21} <4041> of S_{OA2} <404> is utilized for heating of the reboiler V_{SRD} <406>. The remaining portion of S_{OA2} <404> is utilized for heating of the evaporator V_{SA′} <106> at the bottom of the column RR_{A} <100> and of the evaporator V_{SB′} <206> at the bottom of the column RR_{B} <200>.
2) As in FIG. 10, energy in the form of heat is transferred from S_{AP} <104> and from S_{BP} <204> via the heat transferrer WT_{AB2} <418> to a portion of the stream of S_{OA} <302> which is precompressed in the compressor VD_{AB2} <303>. In addition, stream S_{OA11} <4031>, after it has transferred energy in the intermediate evaporator V_{ZRD} <409> to the sidestream S_{ZA} <305>, is likewise directed through the heat transferrer WT_{AB2} <418> in order to transfer the residual energy in S_{OA11} <4031> in the form of heat from S_{OA11} <4031> to the portion of stream S_{OA} <302> that is to be precompressed by means of VD_{AB2} <303>.
3) The following streams are expanded in a condensate vessel <419> (described in section 4.9.5) before they are applied in combination to the rectification column RD_{A} <300>:
the portion of stream S_{OA} <302> which is directed as reflux through the condenser K_{RD} <407>;
S_{OA11} <4031>, after this stream has passed through the intermediate evaporator V_{ZRD} <409> and the heat transferrer WT_{AB2} <418>;
S_{OA2} <404>, after part of this stream has passed through the reboiler V_{SRD} <406> and part through the reboilers V_{SA′} <106> and V_{SB′} <206>;
the optional stream of fresh methanol <408>.
4) It is additionally advantageous to connect the conduit that leads from the condensate vessel <419> to the rectification column RD_{A} <300> to the feed to the condenser K_{RD} <407> in order to establish equalization of pressure. This optional embodiment is shown in dotted form as arrow <420>. In the embodiments in which a reflux is established via <311>, the connection <420> can also be effected from the conduit to <311> that leads from the condensate vessel <419> to the rectification column RD_{A} <300>.

The resultant advantages with regard to energy efficiency and the extension of the lifetime of the compressor VD_{AB2} <303> correspond to those that have been described for the embodiment according to FIG. 10.

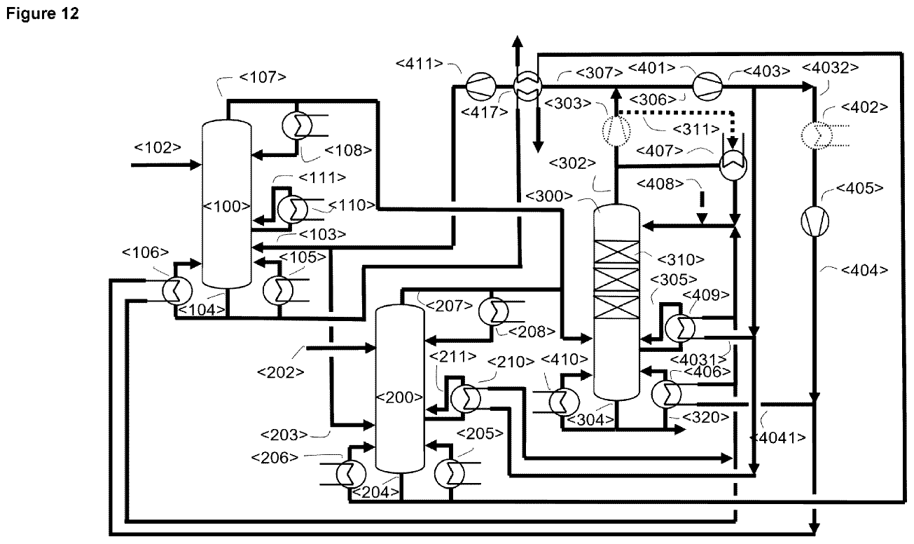

### 3.12 FIG. 12

FIG. 12 shows one embodiment of the process according to the invention. It corresponds to the embodiment described in FIG. 10 with the following differences:
1) The reaction columns RR_{A} <100> and RR_{B} <200> each have an intermediate evaporator V_{ZA} <110> and V_{ZB} <210>. A side stream S_{ZAA} <111> is withdrawn from the reaction column RR_{A} <100> and passed through V_{ZA} <110> before being returned to the reaction column RR_{A} <100>. A side stream S_{ZBA} <211> is withdrawn from the reaction column RR_{B} <200> and passed through V_{ZB} <210> before being returned to the reaction column RR_{B} <200>.
2) Only a portion S_{OA21} <4041> of S_{OA2} <404> is utilized for heating of the reboiler V_{SRD} <406>. The remaining portion of S_{OA2} <404> is utilized for heating of the evaporator V_{SA′} <106> at the bottom of the column RR_{A} <100>.
3) A portion of S_{OA1} <403> is utilized for heating of the intermediate evaporator V_{ZB} <210>.
4) The precompression of the portion of the vapour stream S_{OA} <302> with preliminary compressor VD_{AB2} <303> is optional.
5) The compressor [symbol] <411> is not optional but compulsory.

By means of a heat transferrer WL <417>, energy in the form of heat is transferred from S_{AP} <104> and S_{BP} <204> to [symbol] <307>.

The resultant advantages with regard to energy efficiency and the extension of the lifetime of the compressor [symbol] <411> correspond to those that have been described for the embodiment according to FIG. 10.

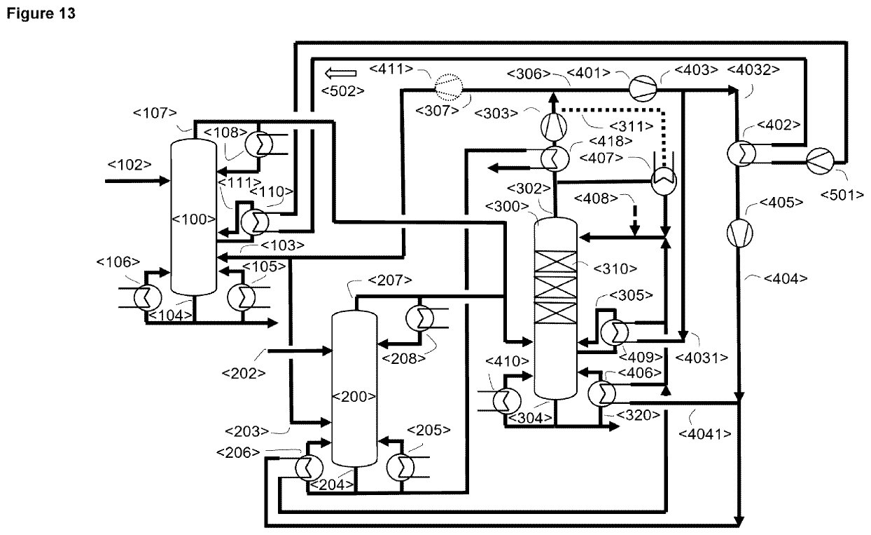

### 3.13 FIG. 13

FIG. 13 shows one embodiment of the process according to the invention. This corresponds to the embodiments described in FIG. 10 with the following differences:
1) The reaction column RR_{A} <100> has an intermediate evaporator V_{ZA} <110>. A side stream S_{ZAA} <111> is withdrawn from the reaction column RR_{A} <100> and passed through V_{ZA} <110> before being returned to the reaction column RR_{A} <100>.
2) Only a portion S_{OA21} <4041> of S_{OA2} <404> is utilized for heating of the reboiler V_{SRD} <406>. The remaining portion of S_{OA2} <404> is used for heating of the evaporator V_{SB′} <206> at the bottom of the column RR_{B} <200>.
3) The intermediate evaporator V_{ZA} <110> is heated by means of a heat transfer medium W₁ <502>, in particular water, which is transported by a pump <501> that absorbs heat from S_{OA12} <4032> in the intermediate cooler WTₓ <402> and releases it in the intermediate evaporator V_{ZA} <110>.
4) By means of the heat transferrer WT_{AB2} <418>, energy is transferred in the form of heat from S_{BP} <204> to a portion of the stream of S_{OA} <302>, but not of S_{AP} <104>.

The resultant energy advantages correspond to those that have been described for the embodiment according to FIG. 10.

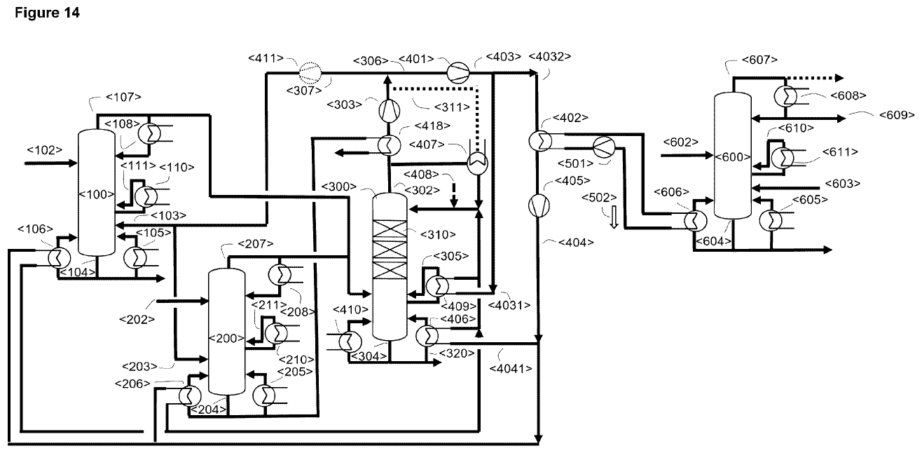

### 3.14 FIG. 14

FIG. 14 shows one embodiment of the process according to the invention. This corresponds to the embodiment described in FIG. 10 with the following differences:
1) The reaction columns RR_{A} <100> and RR_{B} <200> each have an intermediate evaporator V_{ZA} <110> and V_{ZB} <210>. A side stream S_{ZAA} <111> is withdrawn from the reaction column RR_{A} <100> and passed through V_{ZA} <110> before being returned to the reaction column RR_{A} <100>. A side stream S_{ZBA} <211> is withdrawn from the reaction column RR_{B} <200> and passed through V_{ZB} <210> before being returned to the reaction column RR_{B} <200>.
2) Only a portion S_{OA21} <4041> of S_{OA2} <404> is utilized for heating of the reboiler V_{SRD} <406>. The remaining portion of S_{OA2} <404> is used for heating of the evaporator V_{SA′} <106> at the bottom of the column RR_{A} <100> and for heating of the evaporator V_{SB′} <206> at the bottom of the column RR_{B} <200>.
3) By means of the heat transferrer WT_{AB2} <418>, energy is transferred in the form of heat from S_{BP} <204> to a portion of the stream of S_{OA} <302>, but not of S_{AP} <104>.
4) In addition, FIG. 14 shows a reactive rectification column RR_{C} <600> for transalcoholization of sodium methoxide to sodium ethoxide which is at least partly operated with energy from the stream S_{OA12} <4032>. The column RR_{C} <600> comprises the reboilers V_{SC} <605> and V_{SC′} <606>.

Sodium methoxide solution S_{CE1} <602> is reacted here with ethanol S_{CE2} <603> in countercurrent in a reaction column RR_{C} <600> to give sodium ethoxide, and this is withdrawn as an ethanolic solution.

Withdrawn at the bottom of the reaction column RR_{C} <600> is a bottom product stream S_{CP} <604> comprising sodium ethoxide.

At the top of the reaction column RR_{C} <600>, a vapour stream SCB <607> is withdrawn. At least a portion of the vapour stream SCB <607> is condensed in the condenser K_{RRC} <608>, and at least a portion thereof is applied in liquid form to the top of the reaction column RR_{C} <600> as reflux. The vapour stream SCB <607> is withdrawn either in gaseous form upstream of the condenser K_{RRC} <608> (marked by a dashed line) and/or in liquid form downstream of the condenser K_{RRC} <608> as stream <609>.

A side stream S_{ZC} <610> is preferably withdrawn from the reaction column RR_{C} <600>, with transfer of energy to said stream via an intermediate evaporator V_{ZC} <611>, and S_{ZC} <610> may then be recycled into RR_{C} <600>.

The sodium methoxide solution S_{CE1} <602> utilized is preferably at least a portion of the bottom streams S_{AP} <104> and S_{BP} <204> obtained in the reaction columns RR_{A} <100> and RR_{B} <200>.

The reboiler V_{SC′} <606> is heated by means of a heat transfer medium W₁ <502>, in particular water, which is transported by a pump <501> that absorbs heat from S_{OA12} <4032> in the intermediate cooler WTₓ <402> and releases it in the reboiler V_{SC′} <606>.

Alternatively, energy may also be appropriately transferred to the reboiler V_{SC′} <606> or the other reboiler V_{SC} <605> from another stream selected from S_{OA2} <404>, S_{OA11} <4031> and S_{OA1} <403> before separation thereof into S_{OA11} <4031> and S_{OA12} <4032>. Energy may likewise be transferred to the ethanol stream S_{CE1} <603>, the sodium methoxide solution S_{CE1} <602> or the side stream S_{ZC} <610> from at least one of the streams S_{OA1} <403>, S_{OA11} <4031>, S_{OA12} <4032>, S_{OA2} <404>, bottom product stream S_{AP} <104>, bottom product stream S_{BP} <204>, in order to achieve even better energy integration.

The energy advantages that arise from the integration of the energy from S_{AP} <104> to the portion of the stream S_{OA} <302> to be precompressed in the compressor VD_{AB2} <303> and the resultant conservation of the compressor apparatus VD_{AB2} <303> correspond to those that have been described for the embodiment according to FIG. 10.

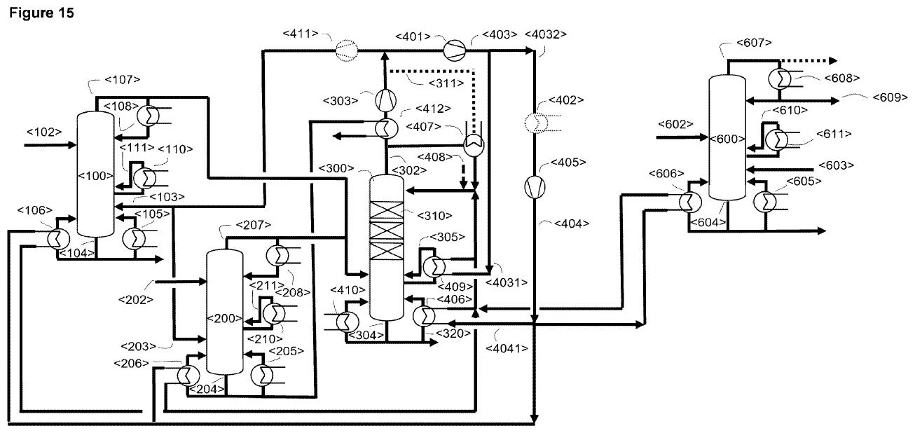

### 3.15 FIG. 15

FIG. 15 shows one embodiment of the process according to the invention. This corresponds to the embodiment described in FIG. 14 with the difference that the heating of the reboiler V_{SC′} <606> is effected directly with a portion of S_{OA2} <404>.

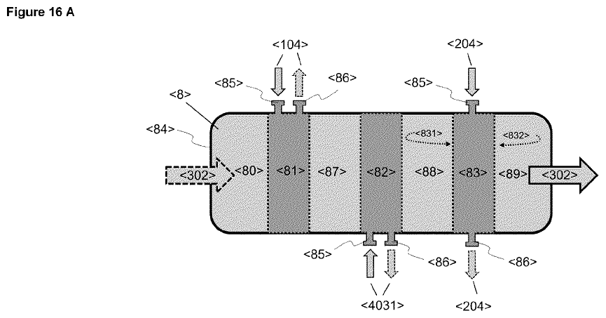

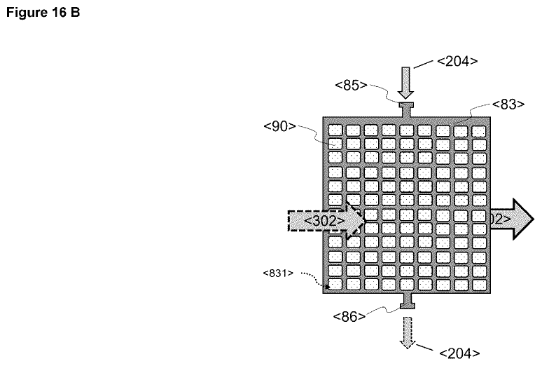

### 3.16 FIGS. 16A and 16B

FIG. 16A shows an embodiment of a heat transferrer <8> as usable, for example, as heat transferrer WT_{AB2} <418> in the embodiment of the invention according to FIG. 11. This comprises three plates <81>, <82> and <83>, which are surrounded by a vessel <84> and which separate the regions <80>, <87>, <88> and <89> from one another.

The plates <81>, <82> and <83> are hollow bodies through which the bottom product streams S_{AP} <104> (plate <81>) and S_{BP} <204> (plate <82>) and the stream S_{OA11} <4031> (plate <83>) are passed. This involves directing the respective stream into the respective plate through a valve <85> and removing it from the respective plate through a valve <86>.

The gaseous stream to be heated, which is the vapour stream S_{OA} <302> in the case shown, is directed, for example, via a valve into the region <80> of the vessel <84> and passes through the plates <81>, <82> and <83> or the regions <80>, <87>, <88> and <89>. The heated gaseous stream, which is the vapour stream S_{OA} <302> in the case shown, then leaves the region <89> of the vessel <84> again, for example via a valve.

The plates <81>, <82> and <83> are arranged in the vessel <84> and/or designed in such a form that the vapour stream S_{OA} <302> can pass through them and, at the same time, energy can be exchanged between S_{OA} <302> and the stream S_{AP} <104>, S_{BP} <204>, S_{OA11} <4031> that passes through the respective plate. For example, the plates <81>, <82> and <83> may be hollow bodies with perforations <90> (holes, for example slots etc.) through which the stream S_{OA} <302> flows.

Of course, these perforations <90> must be fitted such that the medium flowing through the plates <81>, <82> and <83> does not escape.

This embodiment is elucidated in detail in FIG. 16B by the plate <83>. The plate <83> is shown laterally in FIG. 16A, with stream S_{OA} <302> flowing from the side <831> to the side <832>. FIG. 16B shows the plate <83> in top view onto the side <831>.

Alternatively, plates <81>, <82> and <83> may also be used without perforations <90>. But these in that case have to be inserted into the vessel <84> such that the stream to be heated can flow past the plates <81>, <82> and <83> within the vessel; for example, in an oblique manner, i.e., such that the maximum extent thereof is less than the internal diameter of the vessel <84>. In the case of shell-and-tube evaporators, finally, tubes or tube bundles are used instead of the plates <81>, <82> and <83>.

According to the invention, in the embodiment, energy in the form of heat is transferred from S_{AP} <104>, S_{BP} <204> and S_{OA11} <4031> to S_{OA} <302>. S_{AP} <104>, S_{BP} <204> and S_{OA11} <4031> thus have higher energy when fed into the particular plate, as indicated by the respective arrow with a solid line, than in the case of the diversion, as indicated by the respective arrow with the dotted line. Stream S_{OA} <302> is lower in energy (dotted arrow) when fed into the heat transferrer <8> than in the case of the diversion (arrow with solid line). The two valves <85> and <86> may be disposed on the same side of the vessel, as shown in the case of the plates <81> and <82>, or on opposite sides, as in the case of plate <83>.

It will be apparent that the number of plate packages can be varied depending on the number of streams from which energy are used on the stream to be energized. In FIG. 16A, the vapour stream S_{OA} <302>, by means of the plate packages <81>, <82>, <83>, is energized from the streams S_{AP} <104>, S_{BP} <204> and S_{OA11} <4031>.

Optionally, the heat transferrer <8> may be extended by further plates, for example a fourth plate, if, for instance, additional streams are to be utilized as an energy source. Alternatively, these additional plates (or even one of <81>, <82> or <83>) may be operated with an external heat source, which is particularly advantageous for the startup processes. It is likewise optionally possible for one or two of the plates <81>, <82>, <83> to be omitted if, for example, energy has to be transferred solely from one of the streams S_{AP} <104>, S_{BP} <204> and S_{OA11} <4031> to the vapour stream S_{OA} <302>.

In the embodiment according to FIG. 9 (Inventive Example 4), energy is transferred solely from S_{AP} <104> and S_{OA11} <4031> to S_{OA} <302>, for example. In this embodiment, the last plate <83> and hence the region <88> can be omitted.

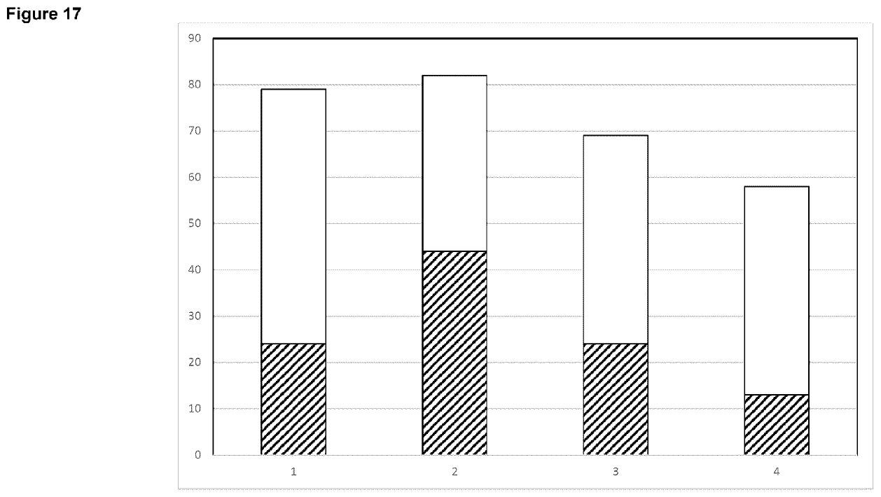

### 3.17 FIG. 17

FIG. 17 illustrates the energy saving in the inventive process according to Example 4 compared to the noninventive processes according to Examples 1 to 3. The x-axis describes the respective example and the y-axis the power to be applied in kilowatts (heating steam and compressor output). The shaded portion of the bars shows the required heating output from low pressure steam while the white portion of the bars shows the sum of the compressor outputs.

### 4. DETAILED DESCRIPTION OF THE INVENTION

The present invention relates to a process for preparing at least one alkali metal methoxide of the formula M_{A}OCH₃, where M_{A} is a metal selected from sodium, potassium, lithium, especially selected from sodium, potassium, and is preferably sodium.

The process according to the invention is conducted in at least one reactive rectification column, and the vapour streams obtained in the at least one reactive rectification column that comprise methanol and water are then separated in a reaction column at least partly into water and methanol. In this distillative separation, there is efficient integration of the energy from the vapours obtained and of the energy from the product stream(s) obtained in the at least one reactive rectification column.

### 4.1 Step (a1)

4.1.1 General description of step (a1)

In step (a1) of the process according to the invention, a reactant stream S_{AE1} comprising methanol is reacted with a reactant stream S_{AE2} comprising M_{A}OH in countercurrent in a reactive rectification column RR_{A} to give a crude product RPA comprising M_{A}OCH₃, water, methanol, M_{A}OH.

According to the invention a “reactive rectification column” is defined as a rectification column in which, at least in some parts, the reaction according to step (a1) or step (a2) of the process according to the invention proceeds. It may also be abbreviated to “reaction column”.

In step (a1), a bottom product stream S_{AP} comprising methanol and M_{A}OCH₃ is withdrawn at the lower end of RR_{A}. A vapour stream S_{AB} comprising water and methanol is withdrawn at the upper end of RR_{A}.

M_{A} is selected from sodium, lithium, potassium. M_{A} is especially selected from sodium, potassium. Preferably, M_{A}=sodium.

The reactant stream S_{AE1} comprises methanol. In a preferred embodiment, the proportion by mass of methanol in S_{AE1} is ≥95% by weight, yet more preferably 99% by weight, and S_{AE1} otherwise comprises especially water.

The methanol used in step (a1) as reactant stream S_{AE1} may also be commercially available methanol having a proportion by mass of methanol of more than 99.8% by weight and a proportion by mass of water of up to 0.2% by weight.

The reactant stream S_{AE1} is preferably introduced in vapour form.

The reactant stream S_{AE2} comprises M_{A}OH. In a preferred embodiment, S_{AE2} comprises not only M_{A}OH but also at least one further compound selected from water, methanol. S_{AE2} more preferably comprises water in addition to M_{A}OH, in which case S_{AE2} is an aqueous solution of M_{A}OH.

When the reactant stream S_{AE2} comprises M_{A}OH and water, the proportion by mass of M_{A}OH, based on the total weight of the aqueous solution forming S_{AE2}, is especially within a range from 10% to 75% by weight, preferably from 15% to 54% by weight, more preferably from 30% to 53% by weight and especially preferably from 40% to 52% by weight.

When the reactant stream S_{AE2} comprises M_{A}OH and methanol, the proportion by mass of M_{A}OH in methanol, based on the total weight of the solution forming S_{AE2}, is especially within a range from 10% to 75% by weight, preferably from 15% to 54% by weight, more preferably from 30% to 53% by weight and especially preferably from 40% to 52% by weight.

In the particular case in which the reactant stream S_{AE2} comprises both water and methanol in addition to M_{A}OH, it is particularly preferable that the proportion by mass of M_{A}OH in methanol and water, based on the total weight of the solution forming S_{AE2}, is especially within a range from 10% to 75% by weight, preferably from 15% to 54% by weight, more preferably from 30% to 53% by weight and particularly preferably from 40% to 52% by weight.
Step (a1) is conducted in a reactive rectification column (or “reaction column”) RR_{A}.
Step (a2), which is elucidated in detail in section 4.2, is likewise performed in a reactive rectification column (or “reaction column”) RR_{B}.

The reaction column RR_{A}/RR_{B} preferably contains internals. Suitable internals are, for example, trays, structured packings or unstructured packings. When the reaction column RR_{A}/RR_{B} contains trays, then bubble cap trays, valve trays, tunnel-cap trays, Thormann trays, cross-slit bubble cap trays or sieve trays are suitable. When the reaction column RR_{A}/RR_{B} contains trays it is preferable to choose trays where not more than 5% by weight, more preferably less than 1% by weight, of the liquid trickles through the respective trays. The construction measures required to minimize trickle-through of the liquid are familiar to those skilled in the art. In the case of valve trays, for example, particularly tightly closing valve designs are selected. Reducing the number of valves also makes it possible to increase the vapour velocity in the tray openings to twice the value typically established. When sieve trays are used, it is particularly favourable to reduce the diameters of the tray openings and to maintain or even increase the number of openings.

When structured or unstructured packings are used, structured packings are preferred in terms of uniform distribution of the liquid.

For columns comprising unstructured packings, especially comprising random packings, and for columns comprising structured packings, the desired characteristics of the liquid distribution may be achieved when the liquid trickling density in the edge region of the column cross section adjacent to the column shell, corresponding to about 2% to 5% of the total column cross section, is reduced compared to the other cross-sectional regions by up to 100%, preferably by 5% to 15%.

This can easily be achieved by, for example, targeted distributions of the drip points of the liquid distributors or the holes thereof.

The process according to the invention can be performed either continuously or batchwise. It is preferably performed continuously.

According to the invention, the “reaction of a reactant stream S_{AE1} comprising methanol with a reactant stream S_{AE2} comprising M_{A}OH in countercurrent” is achieved more particularly by virtue of the feed point for at least a portion of the reactant stream S_{AE1} comprising methanol in step (a1) being below the feed point of the reactant stream S_{AE2} comprising M_{A}OH in the reaction column RR_{A}.

The reaction column RR_{A} preferably comprises at least 2, in particular 15 to 40, theoretical plates between the feed point of the reactant stream S_{AE1} and the feed point of the reactant stream S_{AE2}.

The reaction column RR_{A} may be operated as a pure stripping column. In that case, the reactant stream S_{AE1} comprising methanol is introduced in vapour form in the lower region of the reaction column RR_{A}.

Step (a1) also encompasses the case where a portion of the reactant stream S_{AE1} comprising methanol is added in vapour form below the feed point of the reactant stream S_{AE2} comprising M_{A}OH but nevertheless at the upper end or in the region of the upper end of the reaction column RR_{A}. This makes it possible to reduce the dimensions of the lower region of the reaction column RR_{A}. When a portion of the reactant stream S_{AE1} comprising methanol is added, especially in vapour form, at the upper end or in the region of the upper end of the reaction column RR_{A}, only a fraction of 10% to 70% by weight, preferably of 30% to 50% by weight, (based in each case on the total amount of methanol used in step (a1)) is introduced at the lower end of the reaction column RR_{A}, and the remaining fraction is added in vapour form in a single stream or divided into a plurality of substreams, preferably 1 to 10 theoretical plates, more preferably 1 to 3 theoretical plates, below the feed point of the reactant stream S_{AE2} comprising M_{A}OH.

In the reaction column RR_{A}, the reactant stream S_{AE1} comprising methanol is then reacted with the reactant stream S_{AE2} comprising M_{A}OH according to the above-described reaction <1> to give M_{A}OCH₃ and H₂O, where these products are present in admixture with the methanol and M_{A}OH reactants since the reaction is an equilibrium reaction. Accordingly, in step (a1), a crude product RPA comprising methanol and M_{A}OH in addition to the M_{A}OCH₃ and water products is obtained in the reaction column RR_{A}.

The bottom product stream S_{AP} comprising methanol and M_{A}OCH₃ is then obtained and withdrawn at the lower end of RR_{A}.

The stream of methanol that still contains water, referred to above as “vapour stream S_{AB} comprising water and methanol”, is withdrawn at the upper end of RR_{A}, preferably at the column top of RR_{A}.

This vapour stream S_{AB} comprising water and methanol is directed in step (a3) at least partly into a rectification column RD_{A}, where it is separated by distillation at least partly into a vapour stream S_{OA} comprising methanol, which is withdrawn at the upper end of RD_{A}, and at least one stream S_{UA} comprising water, which is withdrawn at the lower end of RD_{A}. In the embodiments of the present invention in which step (a2) is conducted, at least a portion of vapour stream S_{BB}, mixed with S_{AB} or separately from S_{AB}, is additionally directed into the rectification column RD_{A}.

A portion of the methanol obtained in stream S_{OA} in distillation in step (a3) can be fed to the reaction column RR_{A} as reactant stream S_{AE1} and, if step (a2) is conducted, can alternatively or additionally in step (a2) be fed to the reaction column RR_{B} as reactant stream S_{BE1}.

In a more preferred embodiment of the process according to the invention, 5% to 95% by weight, preferably 10% to 90% by weight, more preferably 20% to 80% by weight, yet more preferably 30% to 70% by weight, yet more preferably 50% to 60% by weight, yet more preferably 56.7% by weight, of the vapour stream S_{OA} is used as reactant stream S_{AE1} or, if step (a2) is conducted, alternatively or additionally as reactant stream S_{BE1} in step (a2).

In this preferred embodiment, it is advantageous to compress the portion of the stream S_{OA} used as reactant stream S_{AE1}/as reactant stream S_{BE1}.

Embodiments [symbol] , ♥, ♦ that are preferred in this regard are elucidated in section 4.10.

The amount of methanol encompassed by the reactant stream S_{AE1} is preferably chosen such that it simultaneously serves as solvent for the alkali metal methoxide M_{A}OCH₃ obtained in the bottom product stream S_{AP}. The amount of methanol in the reactant stream S_{AE1} is preferably chosen so as to achieve, in the bottom of the reaction column RR_{A}, the desired concentration of the alkali metal methoxide solution which is withdrawn as bottom product stream S_{AP} comprising methanol and M_{A}OCH₃.

In a preferred embodiment of the process according to the invention, and especially in the cases in which S_{AE2} comprises not only M_{A}OH but also water, the ratio of the total weight (mass; unit:kg) of methanol used as reactant stream S_{AE1} in step (a1) to the total weight (mass; unit:kg) of M_{A}OH used as reactant stream S_{AE2} in step (a1) is 4:1 to 50:1, more preferably 8:1 to 48:1, even more preferably 10:1 to 45:1, more preferably 20:1 to 40:1, even more preferably 22:1.

The reaction column RR_{A} is operated with or without, preferably with, reflux. “With reflux” means that the vapour stream S_{AB}/S_{BB} comprising water and methanol withdrawn at the upper end of the respective column, in step (a1) from the reaction column RR_{A}, in the optional step (a2) from the reaction column RR_{B}, is not completely discharged. In that case, in step (a3), the respective vapour stream S_{AB}/S_{BB} is thus not directed in its entirety into a rectification column RD_{A}, but rather at least partly, preferably partly, returned to the respective column as reflux, in step (a1) to the reaction column RR_{A}, and in the optional step (a2) to the reaction column RR_{B}. In the cases where such a reflux is established, the reflux ratio is preferably 0.01 to 1, more preferably 0.02 to 0.9, yet more preferably 0.03 to 0.34, yet more preferably 0.04 to 0.27, yet more preferably 0.05 to 0.24, yet more preferably 0.06 to 0.10, yet more preferably 0.07 to 0.09. Generally and in the context of the present invention, a reflux ratio is understood to mean the ratio of the proportion of the mass flow withdrawn from the column (kg/h) that is recycled into the column in liquid form (reflux) to the proportion of this mass flow (kg/h) that is discharged from the respective column in liquid form or gaseous form.

A reflux can be established by mounting a condenser at the top of the respective column. In step (a1) this is achieved in particular by attaching a condenser K_{RRA} to the reaction column RR_{A}. In step (a2) this is achieved in particular by attaching a condenser K_{RRB} to the reaction column RR_{B}.

In the respective condenser, the respective vapour stream S_{AB}/S_{BB} is at least partly condensed and returned to the respective column, in step (a1) to the reaction column RR_{A}, or in step (a2) to the reaction column RR_{B}.

In the embodiment in which a reflux is established in the reaction column RR_{A}, the M_{A}OH used as reactant stream S_{AE2} in step (a1) may also be at least partly mixed with the reflux stream, and the resulting mixture may be supplied as such to step (a1).

Step (a1) is conducted especially at a temperature within a range from 45° C. to 150° C., preferably within a range from 47° C. to 120° C., more preferably within a range from 60° C. to 110° C., and at a pressure within a range from 0.5 bar abs. to 40 bar abs., preferably within a range from 0.7 bar abs. to 5 bar abs., more preferably within a range from 0.8 bar abs. to 4 bar abs., more preferably within a range from 0.9 bar abs. to 3.5 bar abs., yet more preferably within a range from 1.0 bar abs. to 3 bar abs., yet more preferably at 1.25 bar abs.

In step (a1) of the process according to the invention, a bottom product stream S_{AP} comprising methanol and M_{A}OCH₃ is withdrawn at the lower end of the reaction column RR_{A}.

S_{AP} preferably has a proportion by mass of M_{A}OCH₃ in methanol within a range from 1% to 50% by weight, preferably within a range from 5% to 35% by weight, more preferably within a range from 15% to 35% by weight, most preferably within a range from 20% to 35% by weight, based in each case on the total mass of S_{AP}.

The proportion by mass of residual water in S_{AP} is preferably <1% by weight, preferably <0.8% by weight, more preferably <0.5% by weight, based on the total mass of S_{AP}.

The proportion by mass of starting material M_{A}OH in S_{AP} is preferably <1% by weight, preferably <0.8% by weight, more preferably <0.5% by weight, based on the total mass of S_{AP}.

### 4.1.2 Intermediate Evaporator, Reboiler

In a preferred embodiment, the reaction column RR_{A} comprises at least one evaporator which is especially selected from intermediate evaporators V_{ZA} and reboilers V_{SA}. The reaction column RR_{A} particularly preferably comprises at least one reboiler V_{SA}.

According to the invention, “intermediate evaporators” V_{Z} refer to evaporators disposed above the bottom of the respective column, especially above the bottom of the reaction column RR_{A}/RR_{B} (in that case referred to as “V_{ZA}”/“V_{ZB}”) or above the bottom of the rectification column RD_{A} (in that case referred to as “V_{ZRD}”). In the case of RR_{A}/RR_{B}, what is evaporated therein is especially crude product RP_{A}/RP_{B} which is withdrawn from the column as side stream S_{ZAA}/S_{ZBA}.

According to the invention, “reboilers” Vs refer to evaporators which heat the bottom of the respective column, especially the bottom of the reaction column RR_{A}/RR_{B}, or RR_{C} as used in the preferred embodiment and described in detail further hereinafter (in that case referred to as “V_{SA}” or “V_{SA}”/V_{SB}” or “V_{SB′}”/“V_{SC}” or “V_{SC},”) or the bottom of the rectification column RD_{A} (in that case referred to as “V_{SRD}” or “V_{SRD′}”). In the case of RR_{A}/RR_{B}, what is evaporated therein is especially at least a portion of the bottom product stream S_{AP}/S_{BP}. In the case of RR_{C}, what is evaporated therein is especially bottom product stream S_{CP}. In the case of RD_{A}, in particular, a portion of the bottom product stream S_{UA}, S_{UA}1, is evaporated therein. In the case of the reaction columns RR_{A} and RR_{B}, the proportion by mass of the respective alkali metal methoxide M_{A}OCH₃ in S_{AP} or M_{B}OCH₃ in S_{BP} may be adjusted, especially increased. The desired concentration of M_{A}OCH₃ in S_{AP} or M_{B}OCH₃ in S_{BP} may additionally also be adjusted via the control of the reflux in the respective reaction columns RR_{A} and RR_{B}. The combination of the control via the respective reflux in the reaction column RR_{A} or RR_{B} and via the respective reboiler V_{SA} or V_{SA′}, or V_{SB} or V_{SB′}, is particularly advantageous in order to establish the desired concentration of M_{A}OCH₃ in S_{AP} or M_{B}OCH₃ in S_{BP}.

In another preferred embodiment, it is also possible simply to add fresh methanol to S_{AP} or S_{BP} in order to adjust the concentration of M_{A}OCH₃ in S_{AP} or M_{B}OCH₃ in S_{BP}, especially in order to lower it.

In another preferred embodiment, the reaction column RR_{A} has at least one reboiler V_{SA} through which the bottom product stream S_{AP} is then partly passed, and methanol is partly removed therefrom, which increases the proportion by mass of MAOR in S_{AP}.

This increases the proportion by mass of M_{A}OCH₃ in the bottom product stream S_{AP} while it passes through the reboiler V_{SA}, especially by at least 0.5%, preferably 1%, more preferably 2%, even more preferably 5%. Alternatively, this increases the proportion by mass of M_{A}OCH₃ in the bottom product stream S_{AP} while it passes through the reboiler V_{SA}, especially by 0.5% to 10%, preferably by 1% to 5%, more preferably by 2% to 4%, even more preferably by 2.5% to 3.5%.

An evaporator is typically disposed outside the respective reaction column or rectification column. Since evaporators transfer energy, in particular heat, from one stream to another, they are heat transferrers WT. The mixture to be evaporated is withdrawn via a takeoff from the column and supplied to the at least one evaporator. In the case of the reaction column RR_{A}/RR_{B}, intermediate evaporation of the crude product RP_{A}/RP_{B} involves drawing it off and supplying it to the at least one intermediate evaporator V_{ZA}/V_{ZB}.

In the case of the rectification column RD_{A}, intermediate evaporation involves withdrawing (“drawing off”) at least one side stream S_{ZA} from RD_{A} and supplying it to the at least one intermediate evaporator V_{ZRD}.

In the case of the rectification column RD_{A}, the reboiling involves withdrawing (“drawing off”) at least one stream S_{UA} from RD_{A} and supplying at least a portion, preferably a portion, thereof to the at least one reboiler V_{SRD}.

The evaporated mixture is recycled back into the respective column optionally with a residual proportion of liquid via at least one feed. When the evaporator is an intermediate evaporator, i.e. in particular an intermediate evaporator V_{ZA}/V_{ZB}/V_{ZRD}, the takeoff via which the respective mixture is withdrawn and supplied to the evaporator is a side stream takeoff, and the feed via which the evaporated mixture is returned to the respective column is a side stream feed. When the evaporator is a reboiler, i.e. heats the column bottoms, i.e. is in particular a reboiler V_{SA}/V_{SB}/V_{SRD}, at least a portion of the bottoms takeoff stream, in particular S_{AP}/S_{BP}, is supplied to the reboiler, evaporated and recycled back into the respective column in the region of the column bottom.

However it is alternatively also possible to configure suitable tubes, for example on a suitable tray when using an intermediate evaporator or in the bottom of the respective column, traversed by the heat transfer medium, for example the respective compressed vapour stream S_{OA11}/S_{OA21} (if Vs/V_{Z} are located at the rectification column RD_{A}), or a heating medium W₁ In this case, the evaporation occurs on the tray or in the bottom region of the column. However, it is preferable to arrange the evaporator outside the respective column.

Suitable evaporators that can be used as intermediate evaporators and reboilers include, for example, natural circulation evaporators, forced circulation evaporators, forced circulation flash evaporators, kettle evaporators, falling-film evaporators or thin-film evaporators. Heat exchangers for the evaporator that are typically used in the case of natural circulation evaporators and forced circulation evaporators are a shell-and-tube or plate apparatus. When a shell-and-tube evaporator or plate evaporator is used, the heat transfer medium, for example the compressed vapour stream S_{OA11}/S_{OA21} in V_{SRD}/V_{ZRD} in the rectification column RD_{A} or the heating medium W₁, may either flow through the tubes or plates and the mixture to be evaporated may flow around the tubes, or else the heat transfer medium, for example the compressed vapour stream S_{OA11}/S_{OA21} in V_{SRD}/V_{ZRD} in the rectification column RD_{A} or the heating medium W₁, flows around the tubes or plates and the mixture to be evaporated flows through the tubes or plates. FIG. 16A shows a particular embodiment of a plate package evaporator which can be used in the context of the inventive process as a suitable evaporator, especially as intermediate evaporator and reboiler.

In the case of a falling-film evaporator, the mixture to be evaporated is typically introduced as a thin film on the inside of a tube and the tube is heated externally. In contrast to a falling-film evaporator, a thin-film evaporator additionally comprises a rotor with wipers which distributes the liquid to be evaporated on the inner wall of the tube to form a thin film.

As well as those mentioned, it is also possible to use any desired further evaporator type which is known to those skilled in the art and is suitable for use in a rectification column.

When the evaporator being operated with the compressed vapour stream S_{OA11} or the heating medium W₁ as heating vapour, for example, is an intermediate evaporator, it is preferable when the intermediate evaporator is disposed in the stripping section of the rectification column RD_{A} in the region between the feed point of the vapour stream S_{AB} to be evaporated or of the vapour streams S_{AB} and S_{BB} and above the column bottom and, in the case of the reaction columns RR_{A} and RR_{B}, is disposed below the feed point of the reactant stream S_{AE2} or S_{BE2} and above the column bottom.

This makes it possible to introduce a predominant proportion of the heat energy via the intermediate evaporator. It is thus possible, for example, to introduce more than 80% of the energy via the intermediate evaporator. According to the invention, the intermediate evaporator is preferably arranged and/or configured such that it is used to introduce more than 10%, in particular more than 20%, of the total energy required for the distillation.

Where an intermediate evaporator is used, it is especially advantageous when the intermediate evaporator is arranged such that the respective rectification column/reaction column has 1 to 50 theoretical plates below the intermediate evaporator and 1 to 200 theoretical plates above the intermediate evaporator. In particular, it is preferable when the rectification column/reaction column has 2 to 10 theoretical plates below the intermediate evaporator and 20 to 80 theoretical plates above the intermediate evaporator.

The side stream takeoff via which the mixture from the rectification column/reaction column is supplied to the intermediate evaporator V_{Z} and the side stream feed via which the evaporated mixture from the intermediate evaporator V_{Z} is returned to the respective rectification column/reaction column may be positioned between the same trays of the column. However, it is also possible for the side stream takeoff and side stream feed to be at different heights.

Such an intermediate evaporator V_{ZA} can convert liquid crude product RP_{A} present in the reaction column RR_{A} and comprising M_{A}OCH₃, water, methanol, M_{A}OH at least partly to the gaseous state, preferably partly, thus improving the efficiency of the reaction in step (a1) of the process according to the invention.

Such an intermediate evaporator V_{ZB} can convert liquid crude product RP_{B} present in the reaction column RR_{B} and comprising M_{B}OCH₃, water, methanol, M_{B}OH at least partly to the gaseous state, preferably partly, thus improving the efficiency of the reaction in step (a2) of the process according to the invention.

By virtue of one or more intermediate evaporators V_{ZA}/V_{ZB} being disposed in the upper region of the reaction column RR_{A} or RR_{B}, it is possible to reduce the dimensions in the lower region of the reaction column RR_{A} or RR_{B}. In the embodiment having at least one, preferably two or more, intermediate evaporators V_{ZA}/V_{ZB} it is also possible to introduce substreams of the methanol in liquid form in the upper region of the reaction column RR_{A} or RR_{B}.

### 4.2 Step (a2) (Optional)

### 4.2.1 General Description of Optional Step (a2)

Step (a2) is an optional embodiment of the process according to the invention. This means that step (a2) is or is not conducted in the process according to the invention.

In optional step (a2), simultaneously with and spatially separately from step (a1), a reactant stream S_{BE1} comprising methanol is reacted with a reactant stream S_{BE2} comprising M_{B}OH in countercurrent in a reactive rectification column RR_{B} to give a crude product RP_{B} comprising M_{B}OCH₃, water, methanol, M_{B}OH.

In optional step (a2) of the process according to the invention, a bottom product stream S_{BP} comprising methanol and M_{B}OCH₃ is withdrawn at the lower end of RR_{B}. A vapour stream S_{BB} comprising water and methanol is withdrawn at the upper end of RR_{B}.

M_{B} is selected from sodium, lithium, potassium. M_{B} is especially selected from sodium, potassium. Preferably, M_{B}=potassium.

The reactant stream S_{BE1} comprises methanol. In a preferred embodiment, the proportion by mass of methanol in S_{BE1} is ?95% by weight, yet more preferably 99% by weight, and S_{BE1} otherwise comprises especially water.

The methanol used in the optional step (a2) of the process according to the invention as reactant stream S_{BE1} may also be commercially available methanol having a proportion by mass of methanol of more than 99.8% by weight and a proportion by mass of water of up to 0.2% by weight.

The reactant stream S_{BE1} is preferably introduced in vapour form.

The reactant stream S_{BE2} comprises M_{B}OH. In a preferred embodiment, S_{BE2} comprises not only M_{B}OH but also at least one further compound selected from water, methanol. It is yet more preferable when S_{BE2} also comprises water in addition to M_{B}OH; in that case, S_{BE2} is an aqueous solution of M_{B}OH.

When the reactant stream S_{BE2} comprises M_{B}OH and water, the proportion by mass of M_{B}OH, based on the total weight of the aqueous solution forming S_{BE2}, is especially within a range from 10% to 75% by weight, preferably from 15% to 54% by weight, more preferably from 30% to 53% by weight and especially preferably from 40% to 52% by weight.

When the reactant stream S_{BE2} comprises M_{B}OH and methanol, the proportion by mass of M_{B}OH in methanol, based on the total weight of the solution forming S_{BE2}, is especially within a range from 10% to 75% by weight, preferably from 15% to 54% by weight, more preferably from 30% to 53% by weight and especially preferably from 40% to 52% by weight.

In the particular case in which the reactant stream S_{BE2} comprises both water and methanol in addition to M_{B}OH, it is particularly preferable that the proportion by mass of M_{B}OH in methanol and water, based on the total weight of the solution forming S_{BE2}, is especially within a range from 10% to 75% by weight, preferably from 15% to 54% by weight, more preferably from 30% to 53% by weight and particularly preferably from 40% to 52% by weight.

The optional step (a2) of the process according to the invention is performed in a reactive rectification column (or “reaction column”) RR_{B}. Preferred embodiments of the reaction column RR_{B} are described in section 4.1.1.

According to the invention, the “reaction of a reactant stream S_{BE1} comprising methanol with a reactant stream S_{BE2} comprising M_{B}OH in countercurrent” is achieved more particularly by virtue of the feed point for at least a portion of the reactant stream S_{BE1} comprising methanol in the optional step (a2) being below the feed point of the reactant stream S_{BE2} comprising M_{B}OH in the reaction column RR_{B}.

The reaction column RR_{B} preferably comprises at least 2, in particular 15 to 40, theoretical plates between the feed point of the reactant stream S_{BE1} and the feed point of the reactant stream S_{BE2}.

The reaction column RR_{B} may be operated as a pure stripping column. In that case, the reactant stream S_{BE1} comprising methanol is introduced in vapour form in the lower region of the reaction column RR_{B}.

The optional step (a2) also encompasses the case where a portion of the reactant stream S_{BE1} comprising methanol is added in vapour form below the feed point of the reactant stream S_{BE2} comprising M_{B}OH but nevertheless at the upper end or in the region of the upper end of the reaction column RR_{B}. This makes it possible to reduce the dimensions of the lower region of the reaction column RR_{B}. When a portion of the reactant stream S_{BE1} comprising methanol is added, especially in vapour form, at the upper end or in the region of the upper end of the reaction column RR_{B}, only a fraction of 10% to 70% by weight, preferably of 30% to 50% by weight, (based in each case on the total amount of the methanol used in step (a2)) is fed in at the lower end of the reaction column RR_{B}, and the remaining fraction is added in vapour form in a single stream or divided into a plurality of substreams, preferably 1 to 10 theoretical plates, more preferably 1 to 3 theoretical plates, below the feed point of the reactant stream S_{BE2} comprising M_{B}OH.

In the reaction column RR_{B}, the reactant stream S_{BE1} comprising methanol is then reacted with the reactant stream S_{BE2} comprising M_{B}OH according to the above-described reaction <1> to give M_{B}OCH₃ and H₂O, where these products are present in admixture with the methanol and M_{B}OH reactants since the reaction is an equilibrium reaction. Accordingly, a crude product RP_{B} which contains not only the M_{B}OCH₃ and water products but also methanol and M_{B}OH is obtained in the reaction column RR_{B} in the optional step (a2) of the process according to the invention.

The bottom product stream S_{BP} comprising methanol and M_{B}OCH₃ is then obtained and withdrawn at the lower end of RR_{B}.

The stream of methanol that still contains water, referred to above as “vapour stream S_{BB} comprising water and methanol”, is withdrawn at the upper end of RR_{B}, preferably at the column top of RR_{B}.

This vapour stream S_{BB} comprising water and methanol is directed in step (a3) at least partly into a rectification column RD_{A}, where it is separated by distillation at least partly into a vapour stream S_{OA} comprising methanol, which is withdrawn at the upper end of RD_{A}, and at least one stream S_{UA} comprising water, which is withdrawn at the lower end of RD_{A}. A portion of the methanol obtained in stream S_{OA} in the distillation in step (a3) may be fed to the reaction column RR_{B} as reactant stream S_{BE1}.

In this case, in step (a3) of the process according to the invention, when step (a2) is conducted, at least a portion of the vapour stream S_{BB}, having or having not been mixed with S_{AB} (i.e. in that case separately from S_{AB}), is directed into the rectification column RD_{A}. Preferably, in step (a3) of the process according to the invention, vapour streams S_{BB} and S_{AB} are mixed, and then the mixture is directed into the rectification column RD_{A}.

The amount of methanol encompassed by the reactant stream S_{BE1} is preferably chosen such that it simultaneously serves as solvent for the alkali metal methoxide M_{B}OCH₃ obtained in the bottom product stream S_{BP}. The amount of methanol in the reactant stream S_{BE1} is preferably chosen so as to achieve, in the bottom of the reaction column, the desired concentration of the alkali metal methoxide solution which is withdrawn as bottom product stream S_{BP} comprising methanol and M_{B}OCH₃.

In a preferred embodiment of the process according to the invention, and especially in the cases in which S_{BE2} comprises not only M_{B}OH but also water, the ratio of the total weight (mass; unit:kg) of methanol used as reactant stream S_{BE1} in the optional step (a2) to the total weight (mass; unit:kg) of M_{B}OH used as reactant stream S_{BE2} in the optional step (a2) is 4:1 to 50:1, more preferably 8:1 to 48:1, even more preferably 10:1 to 45:1, yet more preferably 20:1 to 40:1, most preferably 22:1.

The reaction column RR_{B} is operated with or without, preferably with, reflux.

In the embodiment in which a reflux is established in the reaction column RR_{B}, the M_{B}OH used as reactant stream S_{BE2} in the optional step (a2) can also be mixed at least partly with the reflux stream and the resulting mixture can thus be supplied to the optional step (a2).

Optional step (a2) is conducted especially at a temperature within a range from 45° C. to 150° C., preferably within a range from 47° C. to 120° C., more preferably within a range from 60° C. to 110° C., and at a pressure within a range from 0.5 bar abs. to 40 bar abs., preferably within a range from 0.7 bar abs. to 5 bar abs., more preferably within a range from 0.8 bar abs. to 4 bar abs., more preferably within a range from 0.9 bar abs. to 3.5 bar abs., yet more preferably within a range from 1.0 bar abs. to 3 bar abs., most preferably at 1.25 bar abs.

In step (a2) of the process according to the invention, a bottom product stream S_{BP} comprising methanol and M_{B}OCH₃ is withdrawn at the lower end of the reaction column RR_{B}.

S_{BP} preferably has a proportion by mass of M_{B}OCH₃ in methanol within a range from 1% to 50% by weight, preferably within a range from 5% to 35% by weight, more preferably within a range from 15% to 35% by weight, most preferably within a range from 20% to 35% by weight, based in each case on the total mass of S_{BP}.

The proportion by mass of residual water in S_{BP} is preferably <1% by weight, preferably <0.8% by weight, more preferably <0.5% by weight, based on the total mass of S_{BP}.

The proportion by mass of starting material M_{B}OH in S_{BP} is preferably <1% by weight, preferably <0.8% by weight, more preferably <0.5% by weight, based on the total mass of S_{BP}.

### 4.2.2 Intermediate Evaporator, Reboiler

In a preferred embodiment, the reaction column RR_{B} comprises at least one evaporator which is in particular selected from intermediate evaporators V_{ZB} and reboilers V_{SB}. The reaction column RR_{B} particularly preferably comprises at least one reboiler V_{SB} through which the bottom product stream S_{BP} in particular is then directed in part, and methanol is removed partly therefrom, which increases the proportion by mass of M_{B}OR in S_{BP}.

This increases the proportion by mass of M_{B}OCH₃ in the bottom product stream S_{BP} while it passes through the reboiler V_{SB}, especially by at least 0.5%, preferably 1%, more preferably 2%, even more preferably 5%. Alternatively, this increases the proportion by mass of M_{B}OCH₃ in the bottom product stream S_{BP} while it passes through the reboiler V_{SB}, especially by 0.5% to 10%, preferably by 1% to 5%, more preferably by 2% to 4%, even more preferably by 2.5% to 3.5%.

4.2.3 Dividing wall columns (DWC)

In the embodiments of the present invention in which it is performed, step (a2) of the process according to the invention is performed simultaneously with and spatially separately from step (a1). Spatial separation is ensured by performing steps (a1) and (a2) in the two reaction columns RR_{A} and RR_{B}.

In an advantageous embodiment of the invention, the reaction columns RR_{A} and RR_{B} are accommodated in one column shell, where the column is at least partially subdivided by at least one dividing wall. Such a column having at least one dividing wall will be referred to as “DWC”.

Such dividing wall columns are known to the person skilled in the art and are described, for example, in U.S. Pat. No. 2,295,256, EP 0 122 367 A2, EP 0 126 288 A2, WO 2010/097318 A1, WO 2021/148174 A1, WO 2021/148175 A1 and by I. Dejanović, Lj. Matijašević, Ž. Olujić, Chemical Engineering and Processing 2010, 49, 559-580.

CN 105218315 A likewise describes dividing wall columns which are used in the rectification of methanol.

In the dividing wall columns suitable for the process according to the invention, the dividing walls preferably extend to the column floor and, in particular, preferably span at least a quarter, more preferably at least a third, yet more preferably at least half, yet more preferably at least two thirds, yet still more preferably at least three quarters, of the column by height. They divide the columns into at least two reaction spaces in which spatially separate reactions may be carried out. The reaction spaces provided by the at least one dividing wall may be of identical or different sizes.

In this embodiment, the bottom product streams S_{AP} and S_{BP} may be withdrawn separately in the regions separated in each case by the dividing wall and preferably passed through the reboiler V_{SA}/V_{SB} provided for each reaction space formed by the at least one reaction wall, in which methanol can be at least partly removed from S_{AP}/S_{BP}.

In a preferred embodiment of the process according to the invention, accordingly, at least two, more preferably exactly two, of the columns selected from rectification column RD_{A}, reaction column RR_{A} and, if step (a2) is conducted, the reaction column RR_{B} are accommodated in one column shell, in which case the columns are at least partly separated from one another by a dividing wall extending to the bottom of the column.

In the integrated system comprising reaction column RR_{A} (or in the embodiments in which step (a2) is conducted, reaction column RR_{A} and reaction column RR_{B}) and rectification column RD_{A} in the process according to the invention, the rectification column RD_{A} is preferably operated at a pressure selected such that the pressure gradient between the columns is low.

As described above, in an advantageous embodiment of the invention, at least two of the columns selected from rectification column RD_{A}, reaction column RR_{A} and, if step (a2) is conducted, the reaction column RR_{B} are accommodated in one column shell, in which case the columns are each at least partly separated from one another by a dividing wall extending to the bottom of the column.

In the above-described preferred embodiment in which step (a2) is conducted and the process according to the invention is performed in a DWC, the reaction regions corresponding to the reaction columns RR_{A} and RR_{A} and to the rectification column RD_{A} within the column jacket are separated from one another by two dividing walls, where the two dividing walls continue to the base of the column.

In this preferred embodiment, the reaction to give the crude product RP_{A} according to step (a1) or the crude products RP_{A} and RP_{B} according to steps (a1) and (a2) are especially performed in one portion of the DWC, where the reactant stream S_{AE2} and optionally the reactant stream S_{BE2} are added below but at approximately the height of the upper end of the dividing wall, and the reactant stream S_{AE1} and optionally the reactant stream S_{BE1} are added in vapour form at the lower end.

The methanol/water mixture formed above the feed point of the reactant stream is then distributed above the dividing wall over the entire column region which serves as rectifying section of the rectification column RD_{A}. The second/third lower part of the column which has been separated off by the dividing wall is the stripping section of the rectification column RD_{A}. The energy required for the distillation is then supplied via an evaporator at the lower end of the second portion of the column separated by the dividing wall, and this evaporator may be heated conventionally or heated with a portion of the compressed vapour stream S_{OA2}. When the evaporator is conventionally heated an intermediate evaporator heated with a portion of the compressed vapour stream S_{OA11} may additionally be provided.

4.2.4 Fresh methanol

The methanol is consumed in the process according to the invention, and especially in a continuous process regime this therefore has to be replaced by fresh methanol.

The fresh methanol is especially fed directly as reactant stream S_{AE1} comprising methanol into the reaction column RR_{A} or, in the embodiments in which step (a2) is conducted, into the reaction columns RR_{A} and RR_{B}.

In the embodiments in which a portion of the methanol obtained in stream S_{OA} in distillation in step (a3) is fed to the reaction column RR_{A} as reactant stream S_{AE1} and, if step (a2) is conducted, is alternatively or additionally in step (a2) fed to the reaction column RR_{B} as reactant stream S_{BE1}, it is even more preferable when the fresh methanol is fed to the rectification column RD_{A}. In the embodiments [symbol] , ♥, ♦ (elucidated in section 4.10), it is likewise preferable when the fresh methanol is added to the rectification column RD_{A}.

When the fresh methanol is added to the rectification column RD_{A}, it is preferably fed in either in the rectifying section of the rectification column RD_{A} or directly at the top of the rectification column RD_{A}. The optimal feed point depends on the water content of the fresh methanol used, and secondly on the desired residual water content in the vapour stream S_{OA}. The higher the proportion of water in the methanol used, and the higher the purity requirement in the vapour stream S_{OA}, the more advantageous it is to feed it in a few theoretical plates below the top of the rectification column RD_{A}. There are preferably up to 20 theoretical plates and especially 1 to 5 theoretical plates below the top of the rectification column RD_{A}.

When the fresh methanol is added to the rectification column RD_{A}, it is added at the top of the rectification column RD_{A} at temperatures up to boiling point, preferably at room temperature. A dedicated feed may be provided here for the fresh methanol, or else fresh methanol after the condensation and recycling of a portion of the methanol withdrawn at the top of the rectification column RD_{A} may be mixed therewith and be fed together to the rectification column RD_{A}. In this case it is particularly preferable when the fresh methanol is added to a condensate vessel in which the methanol condensed out of the vapour stream S_{OA} is collected (elucidated in detail in section 4.9.5).

### 4.3 Step (a3)

In step (a3) of the process according to the invention, at least a portion of the vapour stream S_{AB}, and, if step (a2) is conducted, at least a portion of the vapour stream S_{BB}, mixed with S_{AB} or separately from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A}.

In the embodiments of the invention in which step (a2) is conducted, it is preferable when, in step (a3), the at least one portion of the vapour stream S_{AB} and the at least one portion of the vapour stream S_{BB} are mixed and then directed into a rectification column RD_{A}. S_{AB} and S_{BB} may alternatively also be directed into the rectification column RD_{A} at two different feed points.

In step (a3) of the process according to the invention, at least a portion of the vapour stream S_{AB}, and, if step (a2) is conducted, at least a portion of the vapour stream S_{BB}, mixed with S_{AB} or separately from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A}.

“At least one vapour stream S_{OA} comprising methanol which is withdrawn at the upper end of RD_{A}” means that the vapour obtained at the upper end of RD_{A} may be withdrawn there as one or more vapour streams. If said stream is withdrawn there in more than one vapour stream, the m vapour streams are referred to as “vapour stream S_{OAI}”, “vapour stream SOAII”, [ . . . ], “vapour stream SOAm”, where “m” indicates the number of vapour streams withdrawn at the upper end of RD_{A} (in Roman numerals).

“At least one stream S_{UA} comprising water which is withdrawn at the lower end of RD_{A}” means that water obtained at the lower end of RD_{A} may be withdrawn there as one or more streams. If said stream is withdrawn there in more than one stream, the n streams are referred to as “stream S_{UAI}”, “stream S_{UAII}”, [ . . . ], “stream S_{UAn}”, where “n” is the number of streams withdrawn at the lower end of RD_{A} (in Roman numerals).

The at least one portion of vapour stream S_{AB} and, when step (a2) is conducted, the at least one portion of vapour stream S_{BB} may be directed into the rectification column RD_{A} via one or more feed points. They are introduced via two or more feed points, for example, in the embodiments in which step (a2) is conducted in the process according to the preferred aspect of the invention and, in step (a3), at least a portion of the vapour stream S_{BB} is used separately from S_{AB}. In this embodiment, the at least one portion of vapour stream S_{AB} and the at least one portion of vapour stream S_{BB} are accordingly directed into the rectification column RD_{A} as two separate streams.

In the embodiments of the present invention in which the at least one portion of the vapour stream S_{AB} and, when step (a2) is conducted, the at least one portion of the vapour stream S_{BB} is/are directed into the rectification column RD_{A} as two or more separate streams, it is advantageous when the feed points for the individual streams are at essentially the same height on the rectification column RD_{A}.

In a preferred embodiment of step (a3) of the process according to the invention, the at least one portion of vapour stream S_{AB}, and, when step (a2) is conducted, the at least one portion of vapour stream S_{BB} are separated in the rectification column RD_{A} into a vapour stream S_{OA} comprising methanol, which is withdrawn at the upper end of RD_{A}, and a stream S_{UA} comprising water, which is withdrawn at the lower end of RD_{A}.

Another term for “upper end” of a rectification column is “top”.

Another term for “lower end” of a rectification column is “bottom” or “foot”.

The pressure of the at least one vapour stream S_{OA} is referred to as “p_{OA}” and its temperature as “T_{OA}”. This relates especially to the pressure and temperature of the at least one vapour stream S_{OA} when it is withdrawn from the rectification column RD_{A} in step (a3).

The pressure p_{OA} is especially within a range from 0.5 bar abs. to 8 bar abs., more preferably within a range from 0.6 bar abs. to 7 bar abs., more preferably within a range from 0.7 bar abs. to 6 bar abs., yet more preferably within a range from 1 bar abs. to 5 bar abs., yet more preferably within a range from 1 bar abs. to 4 bar abs., yet more preferably within a range from 1.0 bar abs. to 2.0 bar abs., and is most preferably 1.1 bar abs.

The temperature T_{OA} is especially in the range from 45° C. to 150° C., more preferably in the range from 48° C. to 140° C., more preferably in the range from 50° C. to 130° C., yet more preferably in the range from 60° C. to 120° C., yet more preferably in the range from 60° C. to 110° C., yet more preferably in the range from 65° C. to 80° C., most preferably 67° C.

Any desired rectification column known to those skilled in the art may be used as rectification column RD_{A} in step (a3) of the process. The rectification column RD_{A} preferably contains internals. Suitable internals are, for example, trays, unstructured packings or structured packings. Trays used are typically bubble-cap trays, sieve trays, valve trays, tunnel-cap trays or slotted trays. Unstructured packings are generally beds of random packing elements. Random packing elements used are typically Raschig rings, Pall rings, Berl saddles or Intalox® saddles. Structured packings are sold, for example, under the Sulzer Mellapack© trade name. Apart from the internals mentioned, further suitable internals are known to a person skilled in the art and can likewise be used.

Preferred internals have a low specific pressure drop per theoretical plate. Structured packings and random packing elements have, for example, a significantly lower pressure drop per theoretical plate than trays. This has the advantage that the pressure drop in the rectification column RD_{A} remains as low as possible and the mechanical power of the compressor and the temperature of the methanol/water mixture to be evaporated therefore remain low.

When the rectification column RD_{A} contains structured packings or unstructured packings, these may be divided or in the form of an uninterrupted packing. Typically, however, at least two packings are provided:
1) Preferably,
when step (a2) is not conducted: one packing above the feed point of S_{AB};
when step (a2) is conducted and S_{AB} and S_{BB} are mixed and then directed into RD_{A}: one packing above the feed point of the mixture of S_{AB} and S_{BB};
when step (a2) is conducted and S_{AB} and S_{BB} are directed separately into RD_{A}: one packing above the feed points of S_{AB} and S_{BB}.
2) Preferably, moreover:
when step (a2) is not conducted: one packing below the feed point of S_{AB};
when step (a2) is conducted and S_{AB} and S_{BB} are mixed and then directed into RD_{A}: one packing below the feed point of the mixture of S_{AB} and S_{BB};
when step (a2) is conducted and S_{AB} and S_{BB} are directed separately into RD_{A}: one packing below the feed points of S_{AB} and S_{BB}.

It is also possible to provide one packing above the feed point of S_{AB}/S_{AB} and S_{BB} and multiple trays below the feed point of S_{AB}/S_{AB} and S_{BB}. If an unstructured packing is used, for example a random packing, the random packing elements are typically disposed on a suitable support grid (for example sieve tray or mesh tray).

In the respective embodiment, it is preferable that the feed point of S_{AB}/S_{AB} and S_{BB} is in the lower half of the column RD_{A}, i.e. S_{AB}/S_{AB} and S_{BB} are directed into the lower half, preferably the lower third, more preferably the lower quarter, of the column RD_{A}.

In step (a3) of the process according to the invention, the at least one vapour stream S_{OA} comprising methanol is then withdrawn at the upper end of the rectification column RD_{A}. The preferred proportion by mass of methanol in this vapour stream S_{OA} is ?99% by weight, more preferably 99.6% by weight, yet more preferably 99.9% by weight, and the remainder is especially water.

Withdrawn at the lower end of RD_{A} is at least one stream S_{UA} comprising water which may preferably include <1% by weight, more preferably <5000 ppmw, yet more preferably 2000 ppmw of methanol.

The withdrawal of the at least one vapour stream S_{OA} comprising methanol at the top of the rectification column RD_{A} in the context of the present invention means more particularly that the at least one vapour stream S_{OA} is withdrawn above the internals in the rectification column RD_{A} as a top stream or as a side stream takeoff.

The withdrawal of the at least one stream S_{UA} comprising water at the bottom of the rectification column RD_{A} in the context of the present invention means more particularly that the at least one stream S_{UA} is withdrawn as bottom stream or at the lower tray of the rectification column RD_{A}.

The rectification column RD_{A} is operated with or without, preferably with, reflux.

“With reflux” means that the vapour stream S_{OA} withdrawn at the upper end of the rectification column RD_{A} is not completely discharged but rather partly condensed and returned to the respective rectification column RD_{A}. In the cases where such a reflux is established, the reflux ratio is preferably 0.0001 to 10, more preferably 0.1 to 5, yet more preferably 0.5 to 2, yet more preferably 0.7 to 1, yet more preferably 0.76.

A reflux may be established by mounting a condenser K_{RD} at the top of the rectification column RD_{A}. The vapour stream S_{OA} is partly condensed in the condenser K_{RD} and returned to the rectification column RD_{A}.

### 4.4 Step (b)

In step (b) of the process according to the invention at least a portion of the at least one vapour stream S_{OA} (“at least a portion of the at least one vapour stream S_{OA}”=“at least a portion of S_{OA}”) is compressed. This portion of the at least one vapour stream S_{OA} which is compressed in step (b) is referred to as “ [symbol] ”. This affords a vapour stream S_{OA1} which is compressed with respect to S_{OA}.

The pressure of the vapor stream S_{OA1} is referred to as “p_{OA1}” and its temperature as “T_{OA1}”.

The pressure p_{OA1} is higher than p_{OA}. The precise value of p_{OA1} may be adjusted by those skilled in the art according to the requirements in step (d) provided that the condition p_{OA1}>p_{OA} is met. The quotient of p_{OA1}/p_{OA} (pressures in bar abs. in each case) is preferably in the range from 1.1 to 10, more preferably 1.2 to 8, more preferably 1.25 to 7, most preferably 1.3 to 6.

The temperature T_{OA1} is especially higher than the temperature T_{OA} and the quotient of T_{OA1}/T_{OA} (temperature in ° C. in each case) is preferably in the range from 1.03 to 10, more preferably 1.04 to 9, more preferably 1.05 to 8, more preferably 1.06 to 7, more preferably 1.07 to 6, most preferably 1.08 to 5.

The preferred values of p_{OA1} and T_{OA1} also apply with preference to S_{OA11} and S_{OA12.}

The at least a portion [symbol] of the vapour stream S_{OA} can be compressed in step (b) in any manner known to the person skilled in the art. For example, the compression can be performed mechanically and as a single-stage or multistage compression, preferably a multistage compression. In a multistage compression, it is possible to use two or more compressors of the same type or compressors of different types. A multistage compression can be effected with one or more compressors. The use of single-stage compression or multi-stage compression depends on the compression ratio and thus on the pressure to which the at least a portion [symbol] of the vapour stream S_{OA} is to be compressed.

A suitable compressor in the process according to the invention, especially for compressing the at least a portion [symbol] of the vapour stream S_{OA} to give S_{OA1} in step (b) or of S_{OA12} to give S_{OA2} in step (e), is any suitable compressor known to the person skilled in the art, preferably mechanical compressors with which gas streams can be compressed. Suitable compressors are, for example, single-stage or multistage turbines, piston compressors, screw compressors, centrifugal compressors or axial compressors.

In a multistage compression, compressors suitable for the respective pressure stages to be overcome are used.

In a particular embodiment of step (b), stream S_{OA}, once it has been obtained at the top of the rectification column RD_{A} and, optionally, the reflux to the rectification column RD_{A} has been separated from S_{OA}, is precompressed using a first compressor VD_{AB2}, and then at least a portion [symbol] of the stream S_{OA} thus precompressed is fed to step (b) and especially compressed with the compressor VD₁ to give S_{OA1}.

If embodiment [symbol] is also conducted, it is preferable that a portion [symbol] other than [symbol] is separated from the stream S_{OA} thus precompressed, and it is even more preferably compressed with at least one further compressor V_{D}.

Alternatively or additionally, in this preferred embodiment of step (b), it is also possible to use, rather than the compressor VD_{AB2} which is downstream of the rectification column RD_{A} and in which S_{OA} is precompressed, a compressor VD_{AB1} which is upstream of the rectification column RD_{A} and with which S_{AB}, S_{BB}, or the mixture of S_{AB} and S_{BB} before the respective stream is directed into RD_{A}, is compressed.

When the vapour stream S_{OA} is subjected to multiple compression stages, according to the invention, stream S_{OA1} from S_{OA} is not compressed until the last compressor stage, after which the division of S_{OA1} into stream S_{OA11}, which is used in step (d) according to the invention, and into stream S_{OA12}, which is compressed further in step (e) to give S_{OA2}. This compression which compresses S_{OA} to S_{OA1} is performed in the figures and examples with the compressor VD₁ (referred to in the figures as <401>).

### 4.5 Step (c)

In step (c) of the process according to the invention, at least one side stream S_{ZA} is withdrawn from RD_{A} and recycled back into RD_{A}.

In a preferred embodiment of step (c) of the process according to the invention, one side stream S_{ZA} is withdrawn from RD_{A} and recycled back into RD_{A}.

According to the invention, “side stream S_{ZA} from RD_{A}” means that the stream is withdrawn at a withdrawal point EZA below the top and above the bottom of RD_{A} and in particular additionally recycled back into RD_{A} at a feed point Z_{ZA} (this is the point at which the respective side stream S_{ZA} is recycled back into the rectification column RD_{A}) below the top and above the bottom of RD_{A}.

This means more particularly that the withdrawal point EZA and preferably also the feed point Z_{ZA} of the respective side stream S_{ZA} on the rectification column RD_{A} are below the withdrawal points EOA for all vapour streams S_{OA} withdrawn from RD_{A}, preferably at least 1, more preferably at least 5, yet more preferably at least 10, theoretical plates below the withdrawal point EOA for the vapour stream S_{OA} withdrawn from RD_{A} that has the withdrawal point EOA furthest down the rectification column RD_{A}.

This also means more particularly that the withdrawal point E_{ZA}, and preferably also the feed point Z_{ZA} for the respective side stream S_{ZA} on the rectification column RD_{A} are above the withdrawal points E_{UA} for all streams S_{UA} withdrawn from RD_{A}, preferably at least 1, yet more preferably at least 2, yet more preferably at least 4, theoretical plates above the withdrawal point E_{UA} for the stream S_{UA} that has the withdrawal point E_{UA} furthest up the rectification column RD_{A}.

In the cases in which at least one vapour stream S_{OA} is at least partly recycled back into the rectification column RD_{A} (which is the case when a reflux is established at the rectification column RD_{A} for example), the feed point Z_{OA} (that is the point at which the at least one vapour stream S_{OA} is at least partly recycled back into the rectification column RD_{A}) of the at least one vapour stream S_{OA} is especially also above the withdrawal points E_{ZA} and especially also above the feed points Z_{ZA} for all side streams S_{ZA} withdrawn from RD_{A}, preferably at least 1, more preferably at least 5, yet more preferably at least 10, theoretical plates above the highest point of all withdrawal and feed points for all side streams S_{ZA} withdrawn from RD_{A}.

In the cases in which at least one stream S_{UA} is at least partly recycled into the rectification column RD_{A}, moreover, the feed point Z_{UA} (i.e. the point at which the at least one stream S_{UA} is at least partly recycled into the rectification column RD_{A}) of the at least one stream S_{UA} is below the withdrawal points E_{ZA} and especially also below the feed points Z_{ZA} of all side streams S_{ZA} withdrawn from RD_{A}, preferably at least 1, yet more preferably at least 2, yet more preferably at least 4, theoretical plates below the lowest point of all withdrawal and feed points for all side streams S_{ZA} withdrawn from RD_{A}.

The withdrawal point E_{ZA} of the side stream S_{ZA} and the feed point Z_{ZA} of the side stream S_{ZA} on the rectification column RD_{A} may be positioned between the same trays of RD_{A}. However, they may also be at different heights.

In a preferred embodiment of the process according to the invention, the withdrawal point E_{ZA} and preferably also the feed point Z_{ZA} of the at least one side stream S_{ZA} on the rectification column RD_{A} are below the feed point Z_{SAB}, and above the bottom of RD_{A}. It is yet more preferable when the withdrawal point E_{ZA} and preferably also the feed point Z_{ZA} of the at least one side stream S_{ZA} on the rectification column RD_{A} are also below the rectifying section of RD_{A}.

The feed point Z_{SAB} refers to the lowermost feed point of all feed points of S_{AB} into RD_{A}, of all feed points of S_{BB} into RD_{A}, and of all feed points of the mixture of S_{AB} and S_{BB} into RD_{A}.

In a particularly preferred embodiment of the process according to the invention, the withdrawal point E_{ZA} and more preferably also the feed point Z_{ZA} of the at least one side stream S_{ZA} on the rectification column RD_{A} are in the upper ⅘, preferably upper ¾, preferably upper 7/10, more preferably upper ⅔, more preferably upper ½, of the region on the rectification column RD_{A} below the feed point Z_{SAB} and above the uppermost of all withdrawal and feed points for all streams S_{UA} withdrawn from RD_{A}.

It is yet more preferable when the withdrawal point E_{ZA} and preferably also the feed point Z_{ZA} of the at least one side stream S_{ZA} on the rectification column RD_{A} are then also below the rectifying section of RD_{A}.

In a further particularly preferred embodiment of the process according to the invention, rectification column RD_{A} contains a rectifying section, and the withdrawal point E_{ZA} and more preferably also the feed point Z_{ZA} of the at least one side stream S_{ZA} on the rectification column RD_{A} are in the upper ⅘, preferably upper ¾, preferably upper 7/10, more preferably upper ⅔, more preferably upper ½, of the region on the rectification column RD_{A} below the rectifying section and above the uppermost of all withdrawal and feed points for all streams S_{UA} withdrawn from RD_{A}.

### 4.6 Step (d)

In step (d) of the process according to the invention, energy is transferred from a first portion S_{OA11} of the compressed vapour stream S_{OA1} to S_{ZA} before S_{ZA} is recycled to RD_{A}.

In step (d), S_{OA1} is especially first divided into at least two portions S_{OA11} and S_{OA12}. The ratio of the mass flows (in kg/h) of S_{OA11} to S_{OA12} is preferably in the range from 1:99 to 99:1, more preferably in the range from 1:50 to 50:1, yet more preferably in the range from 1:20 to 30:1, yet more preferably in the range from 5:20 to 15:1.

In step (d) of the process according to the invention, energy is transferred from the first portion S_{OA11} to S_{ZA}. Step (d) reduces the energy of S_{OA11}, and so stream S_{OA11} in particular undergoes at least partial condensation.

According to the invention, “transfer of energy” especially means “heating”, i.e. transfer of energy in the form of heat.

“Transfer of energy from a first portion S_{OA11} of the compressed vapour stream S_{OA1} to S_{ZA}” also encompasses the cases in which further portions of S_{OA1} other than S_{OA11} and S_{OA12} are separated off. This is the case, for example, in those embodiments of the invention in which a portion of the vapour stream S_{OA1} other than S_{OA11} and S_{OA12} (referred to as “S_{OAV}”) is separated from S_{OA1} and S_{OA}. is then used in step (a1) as reactant stream S_{AE1} and, if step (a2) is conducted, alternatively or additionally in step (a2) as reactant stream S_{BE1}.

The transfer of energy from S_{OA11} to S_{ZA}, preferably the heating of S_{ZA} by S_{OA11}, is preferably effected directly or indirectly.

“Direct” means that S_{OA11} is contacted with S_{ZA} without mixing of the two streams, and so energy, especially heat, is transferred from S_{OA11} to S_{ZA}.

This may be performed in that S_{OA11} and S_{ZA} are directed through an intermediate evaporator V_{ZRD} in the rectification column RD_{A}, and S_{OA11} heats S_{ZA}.

Heat transferrers used (another term for “heat transferrer”=“heat exchanger”), especially the heat transferrers WTₓ, WT_{Y}, WT_{Z} mentioned hereinafter, may be the heat transferrers, especially evaporators, that are familiar to the person skilled in the art. In step (d) of the process according to the invention, energy, more preferably heat, is in particular transferred from S_{OA11} to S_{ZA} in an intermediate evaporator V_{ZRD}.

“Indirect” means more particularly that S_{OA11} is contacted with a heat transfer medium W₁, preferably by means of at least one heat transferrer WTₓ, where the heat transfer medium is not S_{ZA}, i.e. W₁ is different from S_{ZA}, such that energy, preferably heat, is transferred from S_{OA11} to W₁ without mixing of the two streams, and the heat is then transferred from W₁ to S_{ZA} in that W₁ is contacted with S_{ZA}, with or without mixing of S_{ZA} and W₁, but preferably without mixing. If W₁ and S_{ZA} do not mix, the energy, preferably heat, is transferred in a further heat transferrer WT_{Y} in particular.

In a further embodiment of the process according to the invention, in the case of indirect energy transfer from S_{OA11} to S_{ZA}, in particular heating of S_{ZA} by S_{OA11}, energy, preferably heat, may also first be transferred from S_{OA11} to W₁, preferably by contacting via at least one heat exchanger WTₓ, and then transferred from W₁ to a further heat transfer medium W₂ other than S_{ZA}, preferably by contacting via at least one heat exchanger WT_{Y}. In the last step, heat is then transferred from W₂ to S_{ZA}, with or without mixing of S_{ZA} and W₂, preferably without mixing. If W₂ and S_{ZA} do not mix, the energy, preferably heat, is transferred in a further heat transferrer WT_{Z} in particular.

It will be apparent that still further heat transfer media W₃, W₄, W_{S} etc. may accordingly be utilized in further embodiments of the present invention.

Utilizable heat transfer media W₁ and further heat transfer media W₂, W₃, W₄, W_{S} include any heat transfer media known to those skilled in the art, preferably selected from the group consisting of air; water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium W₁ used is water or air, yet more preferably water.

Salt-water solutions that may be used are also described for example in DE 10 2005 028 451 A1 and WO 2006/134015 A1.

After step (d) S_{OA11} may then be fed back to the rectification column RD_{A}, optionally together with fresh methanol and/or with the reflux for the rectification column RD_{A}. In a preferred embodiment, energy transfer from S_{OA11} is continued, especially after the transfer of energy to S_{ZA}.

In a preferred embodiment of the process according to the invention, energy, preferably heat, is transferred first from S_{OA11} to S_{ZA} according to step (d), especially to the portion of S_{OA} which is sent to a compression, preferably the compression in step (b), and then from S_{OA11} to S_{OA}, where, when the compression in step (b) is conducted in two or more stages, said compression may be the preliminary compression of S_{OA} or the last compression of S_{OA} to give S_{OA1}. This is preferably the first compression to which the stream S_{OA} is subjected once it has left the column RD_{A}. This makes it possible to use a portion of the residual energy/residual heat still stored by S_{OA11} in the process, in this case for heating of S_{OA} to be compressed. In addition, any droplets present in S_{OA} are evaporated and hence input of droplets into the compressor is prevented, which increases its lifetime.

Other, preferred additional sinks for the energy, preferably heat, in S_{OA11} are described further down.

Step (d) of the process according to the invention reflects one aspect of the unexpected effect of the present invention. Rather than the surplus energy obtained on compression of the vapour stream S_{OA} to give the compressed vapour stream S_{OA1} being dissipated unutilized here, it is used in the rectification. This is done by first compressing S_{OA} to give S_{OA1}, which allows optimization to the value for transfer of energy from S_{OA11} to S_{ZA}, and then further compressing a portion S_{OA12} other than S_{OA11} to S_{OA2}. The heat of condensation obtained on further compression of S_{OA12} to give S_{OA2} is introduced into the column in the reboiler. The required additional compressor output is less than the heating steam power saved thereby. The process according to the invention requires less energy than that of the prior art, as shown in examples 1 and 2. The compression to S_{OA2} allows the pressure and temperature of S_{OA2} to be adjusted such that optimal energy transfer from S_{OA2} to S_{UA1}/SUA is possible.

### 4.7 Step (e)

In step (e) of the process of the invention, a portion S_{OA12} of the compressed vapour stream S_{OA1} other than S_{OA11} is subjected to further compression to give a vapour stream S_{OA2} that is compressed relative to S_{OA11}.

It will be apparent that, after performance of step (e), S_{OA2} is also compressed relative to S_{OA12} and S_{OA1}.

The pressure of the vapor stream S_{OA2} is referred to as “p_{OA2}” and its temperature as “T_{OA2}”.

The pressure p_{OA2} is higher than p_{OA1} and the quotient of p_{OA2}/p_{OA1} (pressures each in bar abs.) is preferably in the range from 1.1 to 10, more preferably 1.2 to 8, more preferably 1.25 to 7, most preferably 1.3 to 6.

The temperature T_{OA2} is especially higher than the temperature T_{OA1} and the quotient of T_{OA2}/T_{OA1} (temperature each in ° C.) is preferably in the range from 1.03 to 10, more preferably 1.04 to 9, more preferably 1.05 to 8, more preferably 1.06 to 7, more preferably 1.07 to 6, most preferably 1.08 to 5.

The compressing of S_{OA12} in step (e) may be performed by processes known to those skilled in the art. For instance, the compression may be performed mechanically and as a single-stage or multistage compression, preferably a multistage compression. In a multistage compression, it is possible to use two or more compressors of the same type or compressors of different types. The use of single-stage compression or multi-stage compression depends on the pressure to which the vapour S_{OA12} is to be compressed. A precompression described for S_{OA} in the context of step (b) may also be performed for the compression of S_{OA12} to S_{OA2}, but compression in one stage, i.e. especially using one compressor VDₓ, is especially sufficient in step (e).

### 4.8 Step (f)

In step (f) of the process according to the invention, energy, preferably heat, is transferred from at least a portion S_{OA21} of S_{OA2} to at least a portion S_{UA1} of the at least one stream S_{UA} before S_{UA1} is recycled into RD_{A}.

In step (f) of the process according to the, energy, preferably heat, is preferably transferred from at least a portion S_{OA21} of S_{OA2} to a portion S_{UA1} of the at least one stream S_{UA} before S_{UA1} is recycled into RD_{A}.

Step (f) reduces the energy of the at least a portion S_{OA21} of S_{OA2}, and so stream S_{OA21} in particular undergoes at least partial condensation.

Step (f) of the process according to the invention comprises the following preferred embodiments (f1), (f2), (f3):
(f1) energy is transferred from at least a portion S_{OA21} of S_{OA2} to a portion S_{UA1} of the at least one stream S_{UA}, and S_{UA1} is then recycled into RD_{A};
(f2) energy is transferred from at least a portion S_{OA21} of S_{OA2} to a portion S_{UA1′} of the at least one 40 stream S_{UA}, and a portion S_{UA1} of S_{UA1′} is then recycled into RD_{A};
(f3) energy is transferred from at least a portion S_{OA21} of S_{OA2} to the whole of stream S_{UA}, and then the whole of stream S_{UA} or only a portion S_{UA1} of stream S_{UA}, preferably only a portion S_{UA1} of stream S_{UA}, is recycled into RD_{A}.

The transfer of energy from at least a portion S_{OA21} of S_{OA2} to the at least a portion S_{UA1} of the at least one stream S_{UA}, preferably the heating of the at least a portion S_{UA1} of the at least one stream S_{UA} by at least a portion S_{OA21} of S_{OA2}, is preferably direct or indirect.

“Direct” means that at least a portion S_{OA21} of S_{OA2} is contacted with the at least a portion S_{UA1} of the at least one stream S_{UA} without mixing of the two streams, and so energy, especially heat, is transferred from at least a portion S_{OA2} to the at least a portion S_{UA1} of the at least one stream S_{UA}.

This may be performed in that the at least a portion S_{OA21} of S_{OA2} and the at least a portion S_{UA1} of the at least one stream S_{UA} are directed through a reboiler V_{SRD} in the rectification column RD_{A}, and the at least a portion S_{OA21} of S_{OA2} heats the at least a portion S_{UA1} of the at least one stream S_{UA}.

Heat transferrers used, in particular the heat transferrers WTₓ, WT_{Y}, WT_{Z} mentioned hereinafter, may be the heat exchangers familiar to the person skilled in the art, in particular evaporators. In step (f) of the process according to the invention, the energy, preferably heat, is in particular transferred from the at least a portion of S_{OA2} to the at least a portion S_{UA1} of the at least one stream S_{UA} in a reboiler V_{SRD}.

“Indirect” means more particularly that the at least a portion S_{OA21} of S_{OA2} is contacted with at least one heat transfer medium W₁, preferably by means of at least one heat exchanger WTₓ, where the heat transfer medium is not the at least a portion S_{UA1} of the at least one stream S_{UA}, i.e. W₁ is different therefrom, such that energy, preferably heat, is transferred from the at least a portion S_{OA21} of S_{OA2} to the at least one heat transfer medium W₁ without mixing of the two streams, and the heat is then transferred from W₁ to the at least a portion S_{UA1} of the at least one stream S_{UA} in that W₁ makes contact with the component in question with or without mixing of the at least a portion S_{UA1} of the at least one stream S_{UA} and W₁, but preferably without mixing.

In a further embodiment of the process according to the invention, in the case of indirect energy transfer from the at least a portion S_{OA21} of S_{OA2} to the at least a portion S_{UA1} of the at least one stream S_{UA}, in particular the heating of the at least a portion S_{UA1} of the at least one stream S_{UA} by the at least a portion S_{OA21} of S_{OA2}, energy, preferably heat, may also first be transferred from S_{OA2} to W₁, preferably by contacting via at least one heat exchanger WTₓ, and then transferred from W₁ to a further heat transfer medium W₂ other than the at least a portion S_{UA1} of the at least one stream SUA, preferably by contacting via at least one heat exchanger WT_{Y}. In the last step, heat is then transferred from W₂ to the at least a portion S_{UA}1 of the at least one stream S_{UA}, with or without mixing of the at least a portion S_{UA1} of the at least one stream S_{UA} and W₂, preferably without mixing.

It will be apparent that still further heat transfer media W₃, W₄, W_{S} etc. may accordingly be utilized in further embodiments of the present invention.

Utilizable heat transfer media W₁ and further heat transfer media W₂, W₃, W₄, W_{S} include any heat transfer media known to those skilled in the art, preferably selected from the group consisting of air; water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium W₁ used is water or air, and water is the very most preferred.

Salt-water solutions that may be used are also described for example in DE 10 2005 028 451 A1 and WO 2006/134015 A1.

After step (f) the at least a portion S_{OA21} of S_{OA2} may then be fed back to the rectification column RD_{A}, optionally together with fresh methanol and/or with the reflux for the rectification column RD_{A} and/or with the stream S_{OA11} obtained after performance of step (d). In a preferred embodiment, the streams are collected for this purpose in a condensate vessel as described in section 4.9.5.

In a preferred embodiment, transfer of energy from at least a portion S_{OA21} of S_{OA2} is continued, especially after the transfer of the energy to the at least a portion S_{UA1} of S_{UA}.

In a preferred embodiment of the process according to the invention, energy, more preferably heat, is transferred first from the at least a portion S_{OA21} of S_{OA2} to the at least a portion S_{UA1} of S_{UA} according to step (f) and then from the at least a portion S_{OA21} of S_{OA2} to S_{OA}, especially to the portion of S_{OA} that is sent to a compression, preferably to the compression in step (b), where said compression can be a preliminary compression of S_{OA} or the compression of S_{OA} to give S_{OA1}. This is preferably the first compression to which the stream S_{OA} is subjected once it has left the column RD_{A}. This makes it possible to use a portion of the residual energy/residual heat still stored by the at least a portion S_{OA2} in the process, in this case for heating of S_{OA} to be compressed.

Other, preferred additional sinks for the energy, preferably heat, in the at least a portion of S_{OA2} are described hereinbelow (see section 4.3).

### 4.9 Characterizing Step (q)

As shown in the examples (compare Example 3 to Examples 1 and 2), the utilization of the vapour S_{OA} that has been compressed stepwise in that the compressed vapour S_{OA1} stage (in the form of S_{OA11}) is utilized for the transfer of energy to the side stream S_{ZA} and the more highly compressed vapour stage S_{OA2} compared to S_{OA1} (in the form of the at least a portion S_{OA21} of S_{OA2}) is utilized for the transfer of energy to the at least a portion S_{UA}1 of the bottom stream S_{UA} already leads to a saving of energy. This elevated energy efficiency of the process according to the invention is thus already ensured by the combination of steps (b) to (f).

This energy efficiency is increased by the characterizing step (g) of the process according to the invention.

### 4.9.1 General Comments

In the characterizing step (g) of the process according to the invention, energy, preferably heat, is transferred from at least a portion of S_{AP} to at least a portion of one or more streams S_{XA} and, if step (a2) is conducted, energy, preferably heat, is additionally or alternatively transferred from at least a portion of S_{BP} to at least a portion of one or more streams S_{XB}.

S_{XA} and S_{XB} are each independently selected from the group consisting of S_{OA}, S_{OA1}, S_{OA2}. In particular, S_{XA} and S_{XB} are each independently selected from the group consisting of S_{OA}, S_{OA1.} Preferably, S_{XA} and S_{XB} are each S_{OA}.

In other words, in the characterizing step (g), energy, preferably heat, is transferred from at least a portion of S_{AP} to at least a portion of one or more streams selected from S_{OA}, S_{OA1}, S_{OA2}, preferably selected from S_{OA}, S_{OA1}, and, if step (a2) is conducted, energy, preferably heat, is additionally or alternatively transferred from at least a portion of S_{BP} to at least a portion of one or more streams selected from S_{OA}, S_{OA1}, S_{OA2}, preferably selected from S_{OA}, S_{OA1.}

In a particularly preferred embodiment of the characterizing step (g) of the process according to the invention, energy, preferably heat, is accordingly transferred from at least a portion of S_{AP} to at least a portion of stream S_{OA} and, if step (a2) is conducted, energy, preferably heat, is additionally or alternatively transferred from at least a portion of S_{BP} to at least a portion of stream S_{OA}.

By virtue of step (g) according to the invention, the residual energy, especially the residual heat, in the bottom product stream S_{AP} and/or bottom product stream S_{BP} is not dissipated unutilized, but integrated into the process according to the invention. Thus, the energy balance of the process according to the invention is further improved compared to processes not according to the invention (i.e. those that are conducted without step (g)).

The transfer of energy from at least a portion of S_{AP} to at least a portion of one or more streams S_{XA} or from at least a portion of S_{BP} to at least a portion of one or more streams S_{XB} is preferably direct or indirect.

“Direct” means that at least a portion of S_{AP} or S_{BP} is contacted with at least a portion of one or more streams S_{XA} or S_{XB} without mixing of S_{AP} and S_{XA} or S_{BP} and S_{XB}, such that energy, especially heat, is transferred from at least a portion of S_{AP} to S_{XA} or from S_{BP} to S_{XB}.

This can be performed in that the at least a portion of S_{AP} and at least a portion of one or more streams S_{XA} or S_{BP} and at least a portion of one or more streams S_{XB} are directed through a heat transferrer WT (for example the heat transferrers [symbol] , [symbol] or WT_{♥} that are shown in the figures), and energy is transferred from S_{AP} to S_{XA} or from S_{BP} to S_{XB}, especially S_{XA} is heated via S_{AP} or S_{XB} is heated via S_{BP}.

Heat transferrers used, in particular the heat transferrers WTₓ, WT_{Y}, WT_{Z} mentioned hereinafter or else the heat transferrers WT, WT_{♦}, WT_{♦} or WT_{♥} mentioned above, may be the heat exchangers familiar to the person skilled in the art, in particular evaporators.

“Indirect” means more particularly that the at least a portion of S_{AP} or S_{BP} is contacted with at least one heat transfer medium W₁, preferably by means of at least one heat exchanger WTₓ, where the heat transfer medium W₁ is not S_{OA}, S_{OA1} or S_{OA2}, i.e. W₁ is different therefrom, such that energy, preferably heat, is transferred from the at least a portion of S_{AP} or S_{BP} to the at least one heat transfer medium W₁, without mixing of the two streams. Thereafter, W₁ and the at least a portion of one or more streams S_{XA} or S_{XB} are contacted, preferably by means of at least one heat exchanger WT_{Y}, with or without mixing of the two streams, but preferably without mixing, such that the energy, preferably heat, is transferred from W₁ to S_{XA} or S_{XB}.

In a further embodiment of the process according to the invention, in the case of indirect energy transfer, it is also possible first to transfer energy, preferably heat, from at least a portion of S_{AP} or S_{BP} to W₁, preferably by contacting by means of at least one heat exchanger WTₓ, and then from W₁ to a further heat transfer medium W₂, preferably by contacting by means of at least one heat exchanger WT_{Y}, where the heat transfer media W₁ and W₂ are not S_{OA}, S_{OA1} or S_{OA2}. In the last step, heat is then transferred from W₂ to at least a portion of one or more streams S_{XA} or S_{XB}, with or without mixing of W₂ and S_{XA} or S_{XB}, but preferably without mixing.

It will be apparent that still further heat transfer media W₃, W₄, W_{S} etc. may accordingly be utilized in further embodiments of the present invention.

Utilizable heat transfer media W₁ and further heat transfer media W₂, W₃, W₄, W_{S} include any heat transfer media known to those skilled in the art, preferably selected from the group consisting of air; water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium W₁ used is water or air, and water is the very most preferred.

Salt-water solutions that may be used are also described for example in DE 10 2005 028 451 A1 and WO 2006/134015 A1.

The at least a portion of the one or more streams S_{XA} and the at least a portion of the one or more streams S_{XB} to which energy is transferred from S_{AP} or S_{BP} in step (g) of the process according to the invention may be the entirety of the respective stream S_{OA}, S_{OA1}, S_{OA2} or only a portion of the respective stream S_{OA}, S_{OA1}, S_{OA2.}

In particular, in step (g), the at least a portion of stream S_{XA} and the at least a portion of stream S_{XB} is each independently at least one of the following streams (those underlined are preferred):
the at least a portion [symbol] of S_{OA};
the portion [symbol] of S_{OA}, if the above-described “embodiment [symbol] ” is conducted (described in section 4.10.1);
stream S_{OA1} prior to the removal of S_{OA11} and S_{OA12};
the portion S_{OA11} of stream S_{OA1};
the portion S_{OA12} of stream S_{OA1;}
the portion S_{OA}, of stream S_{OA1}, if the above-described “embodiment ♥” is conducted (described in section 4.10.2);
the at least a portion S_{OA21} of S_{OA2};
the portion S_{OA♦} of stream S_{OA2}, if the above-described “embodiment ♦” is conducted (described in section 4.10.3).
   4.9.2 Transfer of Energy from S_{AP} and/or S_{BP} Before Compression of S_{XA} and/or S_{XB}

In a more preferred embodiment of step (g), the at least a portion of stream S_{XA} is compressed after energy has been transferred thereto from the at least a portion of S_{AP} in step (g) and, if step (a2) is conducted, additionally or alternatively, the at least a portion of stream S_{XB} is compressed after energy has been transferred thereto from the at least a portion of S_{BP} in step (g).

This more preferred embodiment is particularly advantageous since, in this way, not only is the integration of the energy from product stream S_{AP} and/or from product stream S_{BP} into the process assured, but energy, especially heat, is additionally supplied to the vapour to be compressed prior to compression thereof. This converts any liquid present in the vapour to be compressed, typically in the form of droplets, to the gaseous state and/or prevents the unwanted condensation of the vapour before or during the compression. This is advantageous since liquid in the stream to be compressed has an adverse effect on the mechanism of a compressor, and the avoidance hence increases the lifetime thereof.

“At least a portion of S_{AP} and, if step (a2) is conducted, additionally or alternatively at least a portion of S_{BP}” is abbreviated hereinafter to “S_{AP} or S_{BP}”.

In this embodiment, energy, preferably heat, is transferred S_{AP} or S_{BP} to at least a portion of stream S_{XA} or at least a portion of stream S_{XB}, which is compressed in the process according to the invention. At the same time, this energy, preferably heat, is transferred from S_{AP} or S_{BP} to the at least a portion of stream S_{XA} or at least a portion of stream S_{XB} before it is compressed.

In a particular embodiment 4.9.2.1, in step (g), energy is transferred from S_{AP} or S_{BP} to the at least a portion of stream S_{OA}, [symbol] , before it is compressed in step (b). If this compression in step (b) is effected in multiple stages, energy is preferably transferred in step (g) from S_{AP} or S_{BP} to the at least a portion of stream S_{OA}, [symbol] , before this at least a portion of stream S_{OA}, [symbol] , is compressed in the first stage.

In a further particular embodiment 4.9.2.2, in step (g), energy is transferred from S_{AP} or S_{BP} to stream S_{OA12} before it is compressed in step (e). If this compression of S_{OA12} in step (e) is effected in multiple stages, energy is preferably transferred in step (g) from the at least a portion of S_{AP} or S_{BP} to stream S_{OA12} before stream S_{OA12} is compressed in the first stage.

A further particular embodiment 4.9.2.3 is as follows: When, in embodiment [symbol] (described in section 4.10.1), stream [symbol] is compressed after being separated from the vapour stream S_{OA} and before being used in step (a1) as reactant stream S_{AE1} and, if step (a2) is conducted, alternatively or additionally in step (a2) as reactant stream S_{BE1}, it is even more preferable when, in step (g), energy is transferred from S_{AP} or S_{BP} to stream S_{OA}, before it is compressed. If this compression of S_{OA}, is effected in multiple stages, energy is preferably transferred in step (g) from the at least a portion of S_{AP} or S_{BP} to stream [symbol] before stream [symbol] is compressed in the first stage.

A further particular embodiment 4.9.2.4 is as follows: When, in embodiment ♥(described in section 4.10.2), stream S_{OA♥} is compressed after being separated from the vapour stream S_{OA1} and before being used in step (a1) as reactant stream S_{AE1} and, if step (a2) is conducted, alternatively or additionally in step (a2) as reactant stream S_{BE1}, it is even more preferable when, in step (g), energy is transferred from S_{AP} or S_{BP} to stream S_{OA♥} before it is compressed. If this compression of S_{OA♥} is effected in multiple stages, energy is preferably transferred in step (g) from at least a portion of S_{AP} or S_{BP} to stream S_{OA♥} before stream S_{OA♥} is compressed in the first stage.

A further particular embodiment 4.9.2.5 is as follows: When, in embodiment ♦ (described in section 4.10.3), stream S_{OA♦} is compressed after being separated from the vapour stream S_{OA2} and before being used in step (a1) as reactant stream S_{AE1} and, if step (a2) is conducted, alternatively or additionally in step (a2) as reactant stream S_{BE1}, it is even more preferable when, in step (g), energy is transferred from S_{AP} or S_{BP} to stream S_{OA♦} before it is compressed. If this compression of S_{OA♦} is effected in multiple stages, energy is preferably transferred in step (g) from at least a portion of S_{AP} or S_{BP} to stream S_{OA♦} before stream S_{OA♦} is compressed in the first stage.

In the embodiments of step (g) of the process according to the invention in which S_{XA} and S_{XB} are each independently selected from the group consisting of S_{OA}, S_{OA1}, preference is given to the above embodiments 4.9.2.1 to 4.9.2.4.

In the embodiments of step (g) of the process according to the invention in which S_{XA} and S_{XB} are each S_{OA}, preference is given to the above embodiments 4.9.2.1 to 4.9.2.3.

### 4.9.3 Preferred Embodiment Ω

The embodiments of step (g) of the process according to the invention in which S_{XA} and S_{XB} are each S_{OA} are referred to hereinafter as “embodiment Ω”. This is particularly advantageous.

In other words, in embodiment Ω, in step (g) of the process according to the invention, energy, preferably heat, is transferred from at least a portion of S_{AP} to at least a portion of stream S_{OA} and, if step (a2) is conducted, energy, preferably heat, is additionally or alternatively transferred from at least a portion of S_{BP} to at least a portion of stream S_{OA}.

In the case of performance of the process according to the invention as per embodiment Ω, it is possible to even further reduce energy efficiency in that to utilize further the energy, preferably heat, from a portion of streams S_{OA1}, S_{OA2}, as well as the transfer to the side stream S_{ZA} (in the case of portion S_{OA11} of S_{OA1}) according to step (d) or to the at least a portion S_{UA}1 of the bottom stream S_{UA} (in the case of the at least a portion S_{OA21} of S_{OA2}) according to step (f) and hence to make a further contribution to the improvement in the overall energy efficiency of the process according to the invention.

In embodiment Ω, it is accordingly preferable to transfer energy, preferably heat, from at least a portion of a stream selected from S_{OA1}, S_{OA2} to the crude product RP_{A} and, if step (a2) is conducted, alternatively or additionally to the crude product RP_{B}.

“[ . . . ] the crude product RP_{A} and, if step (a2) is conducted, alternatively or additionally the crude product RP_{B} [ . . . ]” is abbreviated hereinafter to “RP_{A} or RP_{B}”.

“Transfer of energy, preferably heat, from at least a portion of S_{OA1} to RP_{A} or RP_{B}” more particularly encompasses the transfer of energy, preferably heat,
(i-1) from S_{OA11} to RP_{A} or RP_{B} before S_{OA11} is used in step (d);
(ii-1) from S_{OA11} to RP_{A} or RP_{B} after S_{OA11} has been used in step (d);
(iii-1) from a portion S_{OA14} of stream S_{OA1} other than S_{OA11}, S_{OA12} and S_{OA}. to RP_{A} or RP_{B}.

Preference is given here to embodiments (ii-1) and (iii-1), and even more preference to embodiment (iii-1).

“Transfer of energy, preferably heat, from at least a portion of S_{OA2} to RP_{A} or RP_{B}” more particularly encompasses the transfer of energy, preferably heat,
(i-2) from at least a portion S_{OA21} of S_{OA2} to RP_{A} or RP_{B} before this at least a portion S_{OA21} of S_{OA2} is used in step (f);
(ii-2) from at least a portion S_{OA21} of S_{OA2} to RP_{A} or RP_{B} after this at least a portion S_{OA21} of S_{OA2} has been used in step (f);
(iii-2) from a portion S_{OA22} of stream S_{OA2} other than S_{OA21} to RP_{A} or RP_{B}.

Preference is given here to embodiments (ii-2) and (iii-2), and even more preference to embodiment (iii-2).

For this purpose, in the preferred execution of embodiment 0, at least a portion of the stream in question, preferably a portion of the stream in question, selected from S_{OA1}, S_{OA2} or a heat transfer medium W₁, to which energy has previously been transferred from the stream in question selected from S_{OA1}, S_{OA2}, is directed through an intermediate evaporator V_{ZA}/V_{ZB} and the energy from the stream in question selected from S_{OA1}, S_{OA2} and W₁ is transferred to the crude product stream drawn off via the side stream takeoff on RR_{A}/RR_{B}, especially in that the at least a portion of the stream in question selected from S_{OA1}, S_{OA2} and W₁ is utilized for heating of the evaporator V_{ZA}/V_{ZB}.

Alternatively, and even more preferably, in embodiment 0, at least a portion of the stream in question, preferably a portion of the stream in question, selected from S_{OA1}, S_{OA2} or a heat transfer medium W₁, to which energy has previously been transferred from the stream in question selected from S_{OA1}, S_{OA2}, is then directed through a reboiler V_{SA}/V_{SB} and the energy from at least a portion of the stream in question selected from S_{OA1}, S_{OA2} and W₁ is transferred to the bottom product stream S_{AP}/S_{BP}, especially in that the stream in question selected from S_{OA1}, S_{OA2} and W₁ is utilized for heating of the evaporator V_{SA}/V_{SB}.

### 4.9.4 Plate Package Evaporator

A particular embodiment of an evaporator that can be used as heat transferrer in the context of the present invention, but is especially advantageously used for transfer of energy within the scope of characterizing step (g), is shell-and-tube evaporators, preferably those comprising plate packages.

An example of such a plate package evaporator is shown in FIGS. 16A and 16B.

### 4.9.5 Condensate Vessel

In a preferred embodiment of the present process, the vapour streams S_{OA11} and S_{OA21} obtained after step (d) and (f) are recycled back into RD_{A}. This is preferably effected in that these are collected in a vessel, especially a condensate vessel. Preferably collected in this condensate vessel are also any fresh methanol to be supplied and any reflux established into RD_{A}. The respective streams may be fed to the vessel in that they are expanded into the vessel through a valve. The combined, optionally condensed vapours can then be directed from the vessel into RD_{A}.

A corresponding embodiment <419> is shown in FIG. 11.

4.10 Recycling of the Vapours from Rectification Column RD_{A} as Reactant Stream S_{AE1} and/or S_{BE1} In a preferred embodiment, a portion of at least one of the vapours selected from S_{OA}, S_{OA1}, S_{OA2} is used as reactant stream S_{AE1} and, if step (a2) is conducted, alternatively or additionally as reactant stream S_{BE1} in step (a2). Even more preferably, a portion of at least one of the vapours S_{OA}, S_{OA1} is used as reactant stream S_{AE1} and, if step (a2) is conducted, alternatively or additionally as reactant stream S_{BE1} in step (a2).

In a more preferred embodiment, a portion of one of the vapours selected from S_{OA}, S_{OA1}, S_{OA2} is used as reactant stream S_{AE1} and, if step (a2) is performed, alternatively or additionally as reactant stream S_{BE1} in step (a2). Even more preferably, a portion of one of the vapours S_{OA}, S_{OA1} is used as reactant stream S_{AE1} and, if step (a2) is conducted, alternatively or additionally in step (a2) as reactant stream S_{BE1}. Even more preferably, a portion of the vapour S_{OA} is used as reactant stream S_{AE1} and, if step (a2) is conducted, alternatively or additionally as reactant stream S_{BE1} in step (a2).

It is thus advantageous, in a particular embodiment of the process according to the invention, to choose at least one of the embodiments [symbol] , ♥, ♦ described below, preferably to choose one of the embodiments [symbol] , ♥ described below, most preferably embodiment [symbol] .

### 4.10.1 . . . At the Stage of the Vapour Stream S_{OA}

In a preferred embodiment of the process according to the invention, a portion of the vapour stream S_{OA} other than [symbol] , [symbol] , is separated from S_{OA} and [symbol] is then used in step (a1) as reactant stream S_{AE1} and, if step (a2) is conducted, alternatively or additionally in step (a2) as reactant stream S_{BE1}. This embodiment is abbreviated to “embodiment [symbol] ”.

Even more preferably, in embodiment [symbol] , stream [symbol] is compressed after the vapour stream S_{OA} has been separated off and before it is used in step (a1) as reactant stream S_{AE1} and, if step (a2) is conducted, alternatively or additionally in step (a2) as reactant stream S_{BE1}.

Stream [symbol] may be compressed by any of the means familiar to the person skilled in the art, as described for the compression of stream [symbol] in part (b) in section 4.4. For example, the compression can be performed mechanically and as a single-stage or multistage compression, preferably a multistage compression. In a multistage compression, it is possible to use two or more compressors of the same type or compressors of different types. A multistage compression can be effected with one or more compressors. The use of single-stage compression or multi-stage compression depends on the compression ratio and thus on the pressure to which [symbol] is to be compressed. Typically, [symbol] is compressed to a pressure level corresponding to that in the reaction column RR_{A} and/or RR_{B} in which [symbol] is to be used as reactant stream S_{AE1} or S_{AE2}.

[symbol] is preferably compressed with at least one compressor VDs and then used in step (a1) as reactant stream S_{AE1} and, if step (a2) is conducted, alternatively or additionally in step (a2) as reactant stream S_{BE1}.

In particular, compression of [symbol] is thus accomplished using at least one compressor [symbol] . A suitable compressor [symbol] is any compressor known to the person skilled in the art, preferably mechanical compressors with which gas streams can be compressed. Suitable compressors are, for example, single-stage or multistage turbines, piston compressors, screw compressors, centrifugal compressors or axial compressors.

### 4.10.2 . . . At the Stage of the Vapour Stream S_{OA1}

In a further preferred embodiment of the process according to the invention, a portion of the vapour stream S_{OA1} other than S_{OA11} and S_{OA12}, S_{OA♥}, is separated from S_{OA1} and S_{OA♥} is then used in step (a1) as reactant stream S_{AE1} and, if step (a2) is conducted, alternatively or additionally in step (a2) as reactant stream S_{BE1}. This embodiment is abbreviated to “embodiment ♥”.

Even more preferably, in embodiment ♥, stream S_{OA♥} is compressed after the vapour stream S_{OA1} has been separated off and before it is used in step (a1) as reactant stream S_{AE1} and, if step (a2) is conducted, alternatively or additionally in step (a2) as reactant stream S_{BE1}.

Stream S_{OA♥} may be compressed by any of the means familiar to the person skilled in the art, as described for the compression of stream [symbol] in part (b) in section 4.4. For example, the compression can be performed mechanically and as a single-stage or multistage compression, preferably a multistage compression. In a multistage compression, it is possible to use two or more compressors of the same type or compressors of different types. A multistage compression can be effected with one or more compressors. The use of single-stage compression or multi-stage compression depends on the compression ratio and thus on the pressure to which S_{OA♥} is to be compressed. Typically, S_{OA♥} is compressed to a pressure level corresponding to that in the reaction column RR_{A} and/or RR_{B} in which S_{OA♥} is to be used as reactant stream S_{AE1} or S_{AE2}.

In particular, at least one compressor VD_{♥} is used for this purpose. A suitable compressor VD_{♥} is any compressor known to the person skilled in the art, preferably mechanical compressors with which gas streams can be compressed. Suitable compressors are, for example, single-stage or multistage turbines, piston compressors, screw compressors, centrifugal compressors or axial compressors.

### 4.10.3 . . . At the Stage of the Vapour Stream S_{OA2}

In a further preferred embodiment of the process according to the invention, a portion of the vapour stream S_{OA2} other than S_{OA21}, S_{OA♦}, is separated from S_{OA2} and S_{OA♦} is then used in step (a1) as reactant stream S_{AE1} and, if step (a2) is conducted, alternatively or additionally in step (a2) as reactant stream S_{BE1}. This embodiment is abbreviated to “embodiment ♦”.

Even more preferably, in embodiment ♦, stream S_{OA♦} is compressed after the vapour stream S_{OA2} has been separated off and before it is used in step (a1) as reactant stream S_{AE1} and, if step (a2) is conducted, alternatively or additionally in step (a2) as reactant stream S_{BE1}.

Stream S_{OA♦} may be compressed by any of the means familiar to the person skilled in the art, as described for the compression of stream [symbol] in part (b) in section 4.4. For example, the compression can be performed mechanically and as a single-stage or multistage compression, preferably a multistage compression. In a multistage compression, it is possible to use two or more compressors of the same type or compressors of different types. A multistage compression can be effected with one or more compressors. The use of single-stage compression or multi-stage compression depends on the compression ratio and thus on the pressure to which S_{OA♦} is to be compressed. Typically, S_{OA♦} is compressed to a pressure level corresponding to that in the reaction column RR_{A} and/or RR_{B} in which S_{OA♦} is to be used as reactant stream S_{AE1} or S_{AE2}.

In particular, at least one compressor VD_{♦} is used for this purpose. A suitable compressor VD_{♦} is any compressor known to the person skilled in the art, preferably mechanical compressors with which gas streams can be compressed. Suitable compressors are, for example, single-stage or multistage turbines, piston compressors, screw compressors, centrifugal compressors or axial compressors.

### 4.11 Preferred Aspect: Process for Transalcoholization of an Alkali Metal Alkoxide

In an advantageous embodiment of the present invention the energy comprised in at least one of the streams S_{OA1}, S_{OA2} is used for operation of other industrial processes. This is advantageous especially in sites hosting integrated systems (chemistry parks, technology parks) where there is always a need for heating. This energy may be advantageously utilized especially in the case of integrated systems comprising two or more plants for alkali metal alkoxide production. Such integrated systems typically also include processes for preparing alkali metal alkoxides by transalcoholization. Such processes for transalcoholization are known to the person skilled in the art and are described, for example, in WO 2021/122702 A1, U.S. Pat. No. 3,418,383 A, DE 27 26 491 A1 and CS 213119 B1.

In a particular aspect of the present invention, in the process according to the present invention, energy is used by at least a portion of a stream selected from S_{OA1}, S_{OA2} in a process for preparing an alkoxide M_{C}OR″, where M_{c}OR′ is reacted with R″OH in said process to give a crude product comprising M_{C}OR″ and R′OH, with or without R″OH.

R′ and R″ here are two different C₁ to C₇ hydrocarbon radicals, where, in a preferred embodiment, the hydrocarbon radical R′ has at least one carbon atom fewer than R″.

The C₁ to C₇ hydrocarbon radicals are especially C₁ to C₇ alkyl radicals.

Yet more preferably, R′ is methyl and R″ is selected from ethyl, n-propyl, iso-propyl, sec-butyl, 2-methyl-2-butyl, tert-butyl, 2-methyl-2-pentyl, 3-methyl-3-pentyl, 3-ethyl-3-pentyl, 2-methyl-2-hexyl, 3-methyl-3-hexyl, especially selected from ethyl, iso-propyl, 2-methyl-2-butyl, 3-methyl-3-pentyl, 3-ethyl-3-pentyl.

Preferably, R′ and R″ are two different C₁ to C₄ hydrocarbon radicals, where, in an even more preferred embodiment, the hydrocarbon radical R′ has at least one carbon atom fewer than R″. The C₁ to C₄ hydrocarbon radicals are especially C₁ to C₄ alkyl radicals.

Yet more preferably, R′ is methyl and R″ is a C₂ to C₄ hydrocarbon radical, more preferably a C₂ to C₄ alkyl radical. Yet more preferably, in that case, R′ is methyl and R″ is selected from ethyl, n-propyl, iso-propyl, sec-butyl.

Most preferably, R′=methyl and R″=ethyl.

Mc is a metal selected from lithium, sodium, potassium, preferably potassium, sodium, more preferably sodium.

The preferred process for transalcoholization using the energy from at least a portion of a stream selected from S_{OA1}, S_{OA2} can be conducted by methods known to the person skilled in the art.

In one embodiment, for example, M_{c}OR′ is mixed with R″OH in a suitable vessel, for example a stirred tank, and the reaction is conducted with supply of energy from at least a portion of a stream selected from S_{OA1}, S_{OA2}. The energy is supplied, for example, via a heat transferrer WT, with which energy is transferred from at least a portion of a stream selected from S_{OA1}, S_{OA2} to the reaction mixture comprising M_{c}OR′ with R″OH. The alcohol R′OH that forms is evaporated, giving the crude product comprising M_{C}OR″, with or without R″OH.

In a preferred aspect of the present invention, in the process according to the present invention, in a reactive rectification column RR_{C}, a reactant stream S_{CE1} comprising M_{c}OR′, with or without R′OH, is reacted in countercurrent with a reactant stream S_{CE2} comprising R″OH to give a crude product RP_{C} comprising M_{C}OR″ and R′OH, wherein a bottom product stream S_{CP} comprising M_{C}OR″ is withdrawn at the lower end of RR_{C} and a vapour stream SCB comprising R′OH is withdrawn at the upper end of RR_{C}, and wherein energy is transferred from at least a portion of a stream selected from S_{OA1}, S_{OA2} to the crude product RP_{C}.

The process according to the preferred aspect of the invention (also referred to hereinafter as “transalcoholization”) is especially performed in a reactive rectification column RR_{C}. Suitable reactive rectification columns include columns described for RR_{A} in section 4.1.1 in the context of step (a1).

The reaction column RR_{C} is operated with or without, preferably with, reflux. If a reflux is established, the vapour SCB in particular is directed partly or completely through a condenser K_{RRC}, and the condensed vapour may then be returned to the reaction column RR_{C} or, when R′=methyl, used as reactant stream S_{AE1} or S_{BE1}. When R′=methyl, it may also be used as fresh methanol stream in RD_{A}.

In the transalcoholization, a bottom product stream S_{CP} comprising M_{C}OR″ is withdrawn at the lower end of RR_{C}. A vapour stream SCB comprising R′OH is withdrawn at the upper end of RR_{C}.

In a preferred embodiment, when R′=methyl, the reactant stream S_{CE1} used, comprising M_{c}OR′ with or without R′OH, is at least a portion of S_{AP}, and, when step (a2) is conducted, alternatively (when M_{A} and M_{B} are different alkali metals or when M_{A} and M_{B} are the same alkali metal, especially when M_{A} and M_{B} are different alkali metals) or additionally (especially when M_{A} and M_{B} are the same alkali metal), at least a portion of S_{BP} is used. More preferably, in that case, R″=ethyl. What accordingly takes place is a transalcoholization of alkali metal methoxide to the corresponding alkali metal ethoxide.

When S_{BP} and S_{AP} comprise the same alkali metal methoxide, these two streams may also be used separately or in mixed form as S_{CE1}, i.e. in particular first mixed and then fed to the column RR_{C} as reactant stream S_{CE1} or fed to the column RR_{C} separately as two reactant streams S_{CE1}.

The reactant stream S_{CE2} comprises R″OH. In a preferred embodiment, the proportion by mass of R″OH in S_{CE2} is ?85% by weight, yet more preferably 90% by weight, where S_{CE2} otherwise includes M_{C}OR″ in particular or another denaturing agent. The alcohol R″OH used as reactant stream S_{CE2} may also be commercially available alcohol having a proportion by mass of alcohol of more than 99.8% by weight and a proportion by mass of water of up to 0.2% by weight.

According to the invention, “reaction of a reactant stream S_{CE1} comprising M_{c}OR′, with or without R′OH, with a reactant stream S_{CE2} comprising R″OH in countercurrent” is achieved more particularly by virtue of the feed point for at least a portion of the reactant stream S_{CE1} comprising M_{c}OR′ being above the feed point of the reactant stream S_{CE2} comprising R″OH in the reaction column RR_{C}.

The reaction column RR_{C} is operated with or without, preferably with, reflux.

In a preferred embodiment, the reaction column RR_{C} comprises at least one evaporator which is in particular selected from intermediate evaporators V_{ZC} and reboilers V_{SC}. The reaction column RR_{C} more preferably comprises at least one reboiler V_{SC}.

In the case of the reaction column RR_{C}, intermediate evaporation comprises withdrawal (“takeoff”) of at least one side stream S_{ZC} from RR_{C} and feeding thereof to the at least one intermediate evaporator V_{ZC}.

In the case of the reaction column RR_{C}, reboiling involves withdrawing (“drawing off”) of at least one stream, for example S_{CP} from RR_{C}, and feeding at least a portion, in the case of S_{CP} preferably a portion, to the at least one reboiler V_{SC}.

Suitable evaporators that may be used as intermediate evaporators and reboilers are described in section 4.2.2.

In the transalcoholization, energy, preferably heat, is transferred from at least a portion of a stream selected from S_{OA1}, S_{OA2} to the crude product RP_{C}. This is preferably accomplished by transfer of energy from at least a portion of a stream selected from S_{OA1}, S_{OA2} to S_{CE1} or S_{CE2} before passage thereof into RR_{C} followed by transfer of energy from S_{CE1}/S_{CE2} to the crude product RP_{C} present in RR_{C}, with which they mix together.

Accordingly energy, preferably heat, from at least a portion of a stream selected from S_{OA1}, S_{OA2}, in particular from at least one stream selected from S_{OA11}, S_{OA12}, S_{OA2}, preferably from at least one stream selected from S_{OA11}, a portion of S_{OA2}, is transferred to the crude product RP_{C}.

“Transfer of energy, preferably heat, from at least a portion of S_{OA1} to the crude product RP_{C}” also encompasses the transfer of energy, preferably heat, from at least one stream selected from S_{OA11}, S_{OA12}, stream S_{OA1} before separation thereof into S_{OA11}, S_{OA12} to the crude product RP_{C}.

In addition, crude product RP_{C} may also be directed through an intermediate evaporator V_{ZC} or a reboiler V_{SC} and, in V_{ZC}/V_{SC}, energy, preferably heat, may be transferred from at least a portion of a stream selected from S_{OA1}, S_{OA2} to the crude product RP_{C}.

In addition, the bottom product stream S_{CP} may also be directed partly through a reboiler V_{SC} and then partly recycled back into RR_{C}, with transfer in V_{SC} of energy, preferably heat, from at least a portion of a stream selected from S_{OA1}, S_{OA2} to the recycled portion of S_{CP} and then, in column RR_{C}, from S_{CP} to the crude product RP_{C} present in the column.

This transfer of energy from at least a portion of a stream selected from S_{OA1}, S_{OA2} to the streams specified is direct or indirect, i.e. without or with heat transfer medium W₁, in accordance with the manner described in section 4.1.4.

The preferred embodiment of the process according to the invention enables efficient use of the energy from S_{OA1}, S_{OA2}, in particular from S_{OA2}, S_{OA11}, S_{OA12}. This reduces the total energy demand.

### 5. EXAMPLES

Noninventive Examples 1 to 3 and Inventive Example 4 were conducted as shown in FIGS. 1 to 3 and 9, without implementation of the optional guiding of the reflux <311> shown in FIGS. 1 to 3 and 9 and of the compression of stream [symbol] <307> with compressor VD_{♥} <411> shown in FIGS. 3 and 9 in the respective Examples 1 to 4.

### 5.1 Example 1 (Noninventive), Corresponding to FIG. 1

A stream of aqueous NaOH (50% by weight) S_{AE2} <102> of 100 kg/h is supplied at 30° C. to the top of a reaction column RR_{A} <100>. A vaporous methanol stream S_{AE1} <103> of 1034.9 kg/h is fed in countercurrent at the bottom of the reaction column RR_{A} <100>. The reaction column RR_{A} <100> is operated at a top pressure of 2.15 bar abs. At the bottom of the column RR_{A} <100>, a virtually water-free product stream S_{AP} <104> of 219.7 kg/h is withdrawn (30% by weight sodium methoxide in methanol). The evaporator V_{SA} <105> of the reaction column RR_{A} <100> introduces about 24 kW of heating output using low-pressure steam. A vaporous methanol-water stream S_{AB} <107> is withdrawn at the top of the reaction column RR_{A} <100> and 80 kg/h thereof are condensed in the condenser K_{RRA} <108> and returned to the reaction column RR_{A} <100> as reflux while the remaining stream of 915.2 kg/h is supplied to a rectification column RD_{A} <300>. The rectification column RD_{A} <300> is operated at a top pressure of 2.0 bar abs. At the bottom of the rectification column RD_{A} <300>, a liquid water stream S_{UA} <304> of 72.2 kg/h is discharged (500 ppmw of methanol). At the top of the rectification column RD_{A} <300> a vaporous methanol stream S_{OA} <302> (2 bar, 83° C.; 200 ppmw of water) of 1903.6 kg/h is withdrawn and 63.9 kg/h thereof are condensed in a condenser K_{RD} <407> while the remaining stream is supplied to a first compressor VD_{AB2} <303> and therein compressed to 2.6 bar abs. The stream is subsequently divided and a stream [symbol] <307> of 1034.9 kg/h is recycled to the reaction column RR_{A} <100>. The remainder [symbol] <306> of 804.8 kg/h is fed to a multistage compression with intermediate cooling. In the compressor VD₁ <401>, the stream is compressed to p_{OA1}=4.8 bar abs. and T_{OA1}=156° C. to obtain stream S_{OA1} <403>. In the subsequent intermediate cooling in the intermediate cooler WTₓ <402> the stream is cooled to 145° C. and about 4.4 kW of heat are removed using cooling water. In the compressor VDₓ <405>, the stream S_{OA1} <403> is finally compressed again to 9.0 bar and 200° C. to obtain stream S_{OA2} <404>. The subsequent condenser which is simultaneously the reboiler V_{SRD} <406> of the rectification column RD_{A} <300> provides the about 238 kW of heating output for the rectification column RD_{A} <300>. The methanol stream <404> that condenses here is mixed together with 191.9 kg/h of fresh methanol (1000 ppmw of water) <408> and the 63.9 kg/h of previously condensed vapours and recycled to the top of the rectification column RD_{A} <300>.

The compressor output adds up to around 55 kW. Together with the 24 kW for the heating steam, this results in a compressor and heating steam power demand of about 79 kW.

### 5.2 Example 2 (Noninventive). Corresponding to FIG. 2

The arrangement in the noninventive Example 2 corresponds to that of Example 1 with the following differences:

The rectification column RD_{A} <300> comprises an intermediate evaporator V_{ZRD} <409>. A liquid stream S_{ZA} <305> at 94° C. is withdrawn here from rectification column RD_{A} <300>. Around 230 kW of heat is transferred thereto in the intermediate evaporator V_{ZRD} <409>, partly evaporating the stream, which is then fed back to the rectification column RD_{A} <300>.

A vaporous methanol stream S_{OA} <302> (200 ppmw of water) of 1887.1 kg/h is withdrawn at the top of the rectification column RD_{A} <300> and 89.4 kg/h thereof are condensed in a condenser K_{RD} <407>. The remaining stream is compressed to 2.6 bar abs. in a first compressor VD_{AB2} <303> as in example 1. A substream of 1034.9 kg/h is then recycled to the reaction column RR_{A} <100>. The remainder of 762.8 kg/h is compressed to 5.6 bar abs. and 168° C. to obtain stream S_{OA1} <403>.

The subsequent condenser which is simultaneously the intermediate evaporator V_{ZRD} <409> of the rectification column RD_{A} <300> provides the about 230 kW of heating output for the rectification column RD_{A} <300>. The thus condensed methanol stream <403> is mixed with 191.9 kg/h of fresh methanol <408> and the 89.4 kg/h of previously condensed vapours and returned to the top of the rectification column RD_{A} <300>. The reboiler V_{SRD} <406> of the rectification column RD_{A} <300> introduces about 20 kW of heating output using low pressure steam. Compared to Example 1, the vapour stream has to be compressed only to 5.6 bar abs. rather than to 9 bar abs. in order to be able to transmit the heat to the evaporator V_{ZRD} <409>, since the boiling temperature in the intermediate evaporator V_{ZRD} <409> is lower than in the reboiler V_{SRD} <406>.

The compressor output thus adds up overall only to around 38 kW (rather than 55 kW), while the heating steam demand increases relative to example 1 to around 44 kW since the reboiler V_{SRD} <406> requires a heating output of 20 kW and this is introduced with heat with the aid of low pressure pressure.

The compressor and heating steam power demands thus sum to about 82 kW.

### 5.3 Example 3 (Noninventive), Corresponding to FIG. 3

The arrangement in Noninventive Example 3 corresponds to that of Examples 1 and 2 with the following differences:

A vaporous methanol stream S_{OA} <302> (200 ppmw of water) of 1898.9 kg/h is withdrawn at the top of the rectification column RD_{A} <300> and 33.9 kg/h thereof are condensed in a condenser K_{RD} <407>. The remaining stream is compressed to 2.6 bar abs. in a first compressor VD_{AB2} <303> as in example 2 and subsequently a substream [symbol] <307> of 1034.9 kg/h is recycled to the reaction column RR_{A} <100>. The remainder [symbol] <306> of 830.1 kg/h is first compressed to 5.6 bar abs. and 169° C. to obtain stream S_{OA1} <403>. A portion S_{OA11} <4031> (761.7 kg/h) of this stream is directed into a downstream condenser which is simultaneously the intermediate evaporator V_{ZRD} <409> of the rectification column RD_{A} <300> and provides about 230 kW of heating output for the rectification column RD_{A} <300>. The other portion S_{OA12} <4032> (68.4 kg/h) is subsequently cooled to about 154° C. in an intermediate cooling in intermediate cooler WTₓ <402> wherein about 0.5 kW of heat are removed via cooling water. Stream S_{OA12} <4032> is subsequently compressed in a further compressor VDₓ <405> to obtain stream S_{OA2} <404> with p_{OA2}=9.0 bar and T_{OA2}=196° C.

The downstream condenser, which is simultaneously the reboiler V_{SRD} <406> of the rectification column RD_{A} <300>, provides about 20 kW of heating output for the rectification column RD_{A} <300>. The methanol streams S_{OA11} <4031> and S_{OA2} <404> condensed in the intermediate evaporator and reboiler V_{ZRD} <409> and V_{SRD} <406> are mixed with 191.9 kg/h of fresh methanol <408> and the 33.9 kg/h of previously condensed vapours and returned to the top of the rectification column RD_{A} <300>.

The compressor output sums to about 42 kW (instead of 55 kW in example 1). Since no low-pressure steam is required for the reboiler V_{SRD} <406>, only about 24 kW has to be provided using heating steam as in Example 1. The sum of the compressor and heating steam power demands thus falls to about 66 kW.

Compared to Example 1, only a smaller vapour stream has to be compressed to 9 bar abs., and a majority of the vapour stream only has to be compressed to 5.6 bar abs. as in Example 2, thus reducing the total compressor output. However, compared to Example 2, steady-state operation does not require any low-pressure steam in the reboiler V_{SRD} <406>, and so the heating steam demand reduces compared to Example 2.

The total energy to be supplied is minimized by the process according to Example 3.

Nevertheless, this embodiment has the disadvantage that the energy present in stream S_{AP} <104> is dissipated unutilized. Stream S_{AP} <104> is obtained with a temperature of 105° C. at the bottom of column RR_{A} <100>. Since this stream S_{AP} <104> is not easily managed and storable at the resultant temperature of 105° C., it first has to be cooled to 50° C. before it can be run into a storage tank. This is typically accomplished in that it is left to cool, as a result of which some of the energy stored therein dissipates unutilized. There is likewise no use of the residual energy in the condensed stream <4031>.

### 5.4 Example 4 (Inventive), Corresponding to FIG. 9

The process utilized for Inventive Example 4 is illustrated in FIG. 9. This corresponds to the process according to Example 3 with the differences that follow.

The portion of the vapour stream S_{OA} <302> which is not recycled as reflux via the condenser K_{RD} <407> to the rectification column RD_{A} <300> is conducted via the heat transferrer WT_{AB2} <418>. This heat transferrer WT_{AB2} <418> consists of a vessel in which multiple plate heat transferrer packages can be installed (cf. FIG. 16A). Two plate packages are installed in WT_{AB2} <418>, in order to make the residual heat from streams S_{AP} <104> and S_{OA1} <4031> utilizable for the system. The condensed stream S_{OA1} <4031> is preferably run through the first plate package <81>, and the bottom stream S_{AP} <104> is preferably run through the second plate package <82>. In both plate packages, a portion of the respective residual heat is transferred to the vapour stream S_{OA} <302>, which is overheated by 10 K (from 67° C. to 77° C.) as a result. Firstly, it is thus possible to prevent droplet formation (via pressure drops) on the suction side of the compressor VD_{AB2} <303>; secondly, overheating in the reaction column RR_{A} <100> is utilized. The heating output of the steam-driven reboiler V_{SA} <105> is reduced by 46% by virtue of the thermal integration of the heat transferrer WT_{AB2} <418>. By virtue of the overheating of the portion of the vapour stream S_{OA} <302> directed through the heat transferrer WT_{AB2} <418>, there is also a rise in the power of the multistage compression in the compressors VD_{AB2} <303> and VD₁ <401> by 3%. In addition, this measure greatly reduces the probability of droplet formation on the suction side of the compressors, which can cause severe damage to the impeller in particular. There is thus an increase in the service life of the multistage compressor. The bottom stream S_{AP} <104> is ultimately cooled by this procedure to 87° C., which distinctly simplifies the handling thereof on storage.

Result: The procedure according to Example 3 of stepwise compression of the vapour stream and hence operating the intermediate evaporator and reboiler with the differently compressed vapour can surprisingly save energy. In addition, the inventive procedure according to Example 4 permits a further saving of energy and, when the energy is transferred from the bottom product stream S_{AP} <104> before the compression of the portion of the vapour stream S_{OA} <302>, enables an extension of the lifetime of the compressors. Moreover, there is simplification of the handling of the bottom product stream S_{AP} <104>.

FIG. 17 shows the comparison of the energy to be expended in the respective examples. It is apparent from this figure that the procedure according to the invention as per Example 4 is the most energy-efficient.

### 6. Abbreviations

| | |
|---|---|
| Reaction column RR_{A} | <100> |
| Reactant stream S_{AE2} comprising, for example, NaOH, LiOH or KOH solution | <102> |
| (aqueous or methanolic) | |
| Reactant stream S_{AE1} comprising methanol | <103> |
| Bottom product stream S_{AP} comprising methanol and sodium methoxide, | <104> |
| potassium methoxide or lithium methoxide | |
| Reboiler V_{SA} | <105> |
| Reboiler V_{SA′} (optional) | <106> |
| Vapour stream S_{AB} | <107> |
| Condenser K_{RRA} | <108> |
| Portion of stream S_{AP} from which no energy is transferred (optional) | <309> |
| Intermediate evaporator V_{ZA} | <110> |
| Side stream S_{ZAA} | <111> |
| Reaction column RR_{B} | <200> |
| Reactant stream S_{BE2} comprising, for example, KOH solution (aqueous or | <202> |
| methanolic) | |
| Reactant stream S_{BE1} comprising methanol | <203> |
| Bottom product stream S_{BP} comprising methanol and potassium methoxide | <204> |
| Reboiler V_{SB} | <205> |
| Reboiler V_{SB′} (optional) | <206> |
| Vapour stream S_{BB} | <207> |
| Condenser K_{RRB} | <208> |
| Intermediate evaporator V_{ZB} | <210> |
| Side stream S_{ZBA} | <211> |
| Rectification column RD_{A} | <300> |
| Vapour stream S_{OA} | <302> |
| Compressor VD_{AB2}; this is the compressor which, in accordance with the | <303> |
| invention, precompresses stream S_{OA} or a portion of stream S_{OA}. At least a | |
| portion S_{OA} [symbol] of stream S_{OA} thus precompressed is then compressed in | |
| compressor VD₁ to give S_{OA1} | |
| Bottom stream S_{ZA} comprising water | <304> |
| Side stream S_{ZA} | <305> |
| S_{OA} [symbol] (portion of S_{OA} which is compressed in compressor VD₁ to give S_{OA1}) | <306> |
| S_{OA} [symbol] : portion of the vapour stream S_{OA} other than S_{OA} [symbol] | <307> |
| S_{OA♥}: portion of the compressed vapour stream S_{OA1} other than S_{OA11} | <308> |
| Internals in RD_{A} | <310> |
| Optional guiding of the reflux of a portion of S_{OA} | <311> |
| S_{UA1} (portion of the bottom stream S_{UA}) | <320> |
| Compressor VD₁; this is the compressor which, in accordance with the | <401> |
| invention, compresses stream S_{OA} or a portion S_{OA} [symbol] of stream S_{OA} to give S_{OA1} | |
| Intermediate cooler WT_{X} (optional, if shown by dotted lines) | <402> |
| Vapour stream S_{OA1} compressed relative to vapour stream S_{OA} | <403> |
| S_{OA11} (portion of the compressed vapour stream S_{OA1} from which energy is | <4031> |
| transferred to side stream S_{ZA}) | |
| S_{OA12} (portion of the compressed vapour stream S_{OA1} which is compressed to | <4032> |
| give the vapour stream S_{OA2}) | |
| S_{OA13} (portion of the compressed vapour stream S_{OA1} which is recycled to the | <4033> |
| column RD_{A} as reflux) | |
| S_{OA2}: vapour stream compressed relative to vapour stream S_{OA1}. Energy is | <404> |
| transferred therefrom, or from a portion S_{OA21} thereof, to the bottom stream | |
| S_{UA}. | |
| Portion S_{OA21} of S_{OA2} | <4041> |
| Compressor VDₓ | <405> |
| Reboiler V_{SRD} | <406> |
| Condenser K_{RD} | <407> |
| Optional stream of fresh methanol | <408> |
| Intermediate evaporator V_{ZRD} | <409> |
| Reboiler V_{SRD′} | <410> |
| Compressor VD [symbol] | <411> |
| Heat transferrer WT [symbol] | <412> |
| Compressor VD_{♥} | <415> |
| Heat transferrer WT_{♥} | <416> |
| Heat transferrer WT [symbol] | <417> |
| Heat transferrer WT_{AB2} | <418> |
| Condensate vessel | <419> |
| Optional pressure equalization | <420> |
| Pump | <501> |
| Heat transfer medium W₁ | <502> |
| Reactive rectification column RR_{C} | <600> |
| Reactant stream S_{CE1} | <602> |
| Reactant stream S_{CE2} | <603> |
| Bottom product stream S_{CP} | <604> |
| Reboiler V_{SC} | <605> |
| Reboiler V_{SC′} | <606> |
| Vapour stream S_{CB} | <607> |
| Condenser K_{RRC} | <608> |
| Condensate of vapour stream S_{CB} | <609> |
| Side stream S_{ZC} | <610> |
| Intermediate evaporator V_{ZC} | <611> |
| Special heat transferrer | <8> |
| Plates (or else “plate package”) | <81>, <82>, |
| | <83> |
| Regions within the vessel | <80>, <87>, |
| | <88>, <89> |
| Side of the plate <83> facing the stream to be heated S_{OA} | <831> |
| Side of the plate <83> remote from the stream to be heated S_{OA} | <832> |
| Vessel | <84> |
| Valves | <85>, <86> |
| Perforation | <90> |

1-21. (canceled)

22. A process for preparing at least one alkali metal methoxide of formula M_{A}OCH₃, wherein M_{A} is a metal selected from sodium, potassium, and lithium, and wherein:
(a1) a reactant stream S_{AE1} comprising methanol is reacted with a reactant stream S_{AE2} comprising M_{A}OH in countercurrent in a reactive rectification column RR_{A} to give a crude product RP_{A} comprising M_{A}OCH₃, water, methanol, and M_{A}OH,
wherein a bottom product stream S_{AP}, comprising methanol and M_{A}OCH₃, is withdrawn at the lower end of RR_{A} and a vapour stream S_{AB} comprising water and methanol is withdrawn at the upper end of RR_{A};
(a2) and optionally, simultaneously with and spatially separate from step (a1), a reactant stream S_{BE1} comprising methanol is reacted with a reactant stream S_{BE2} comprising M_{B}OH in countercurrent in a reactive rectification column RR_{B} to give a crude product RP_{B} comprising M_{B}OCH₃, water, methanol, and M_{B}OH, wherein M_{B} is selected from sodium, lithium, and potassium,
wherein a bottom product stream S_{BP} comprising methanol and M_{B}OCH₃ is withdrawn at the lower end of RR_{B} and a vapour stream S_{BB} comprising water and methanol is withdrawn at the upper end of RR_{B};
(a3) at least a portion of the vapour stream S_{AB}, and, if step (a2) is conducted, at least a portion of the vapour stream S_{BB}, mixed with S_{AB} or separately from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A};
(b) at least a portion of S_{OA}, [symbol] , is compressed to give a vapour stream S_{OA1} which is compressed relative to S_{OA};
(c) at least one side stream S_{ZA} is withdrawn from RD_{A} and recycled back into RD_{A};
(d) energy is transferred from a first portion S_{OA11} of the compressed vapour stream S_{OA1} to S_{ZA} before S_{ZA} is recycled into RD_{A};
(e) a portion of the compressed vapour stream S_{OA1} other than S_{OA11}, S_{OA12} is compressed further to give a vapour stream S_{OA2} which is compressed relative to S_{OA11};
(f) energy is transferred from at least a portion S_{OA21} of S_{OA2} to at least a portion S_{UA1} of S_{UA} before S_{UA}1 is recycled into RD_{A};
wherein:
(g) energy is transferred from at least a portion of S_{AP} to at least a portion of one or more streams S_{XA} and, if step (a2) is conducted, energy is additionally or alternatively transferred from at least a portion of S_{BP} to at least a portion of one or more streams S_{XB};
wherein S_{XA} and S_{XB} are each independently selected from the group consisting of S_{OA}, S_{OA1}, and S_{OA2.}

23. The process of claim 22, wherein in step (d), energy is transferred from S_{OA11} to S_{ZA} in an intermediate evaporator V_{ZRD}.

24. The process of claim 22, wherein in step (f), energy is transferred from at least a portion S_{OA21} of S_{OA2} to the at least a portion S_{UA}1 of S_{UA} in a reboiler V_{SRD}.

25. The process of claim 22, wherein energy is transferred first from S_{OA11} to S_{ZA} according to step (d) and then from S_{OA11} to S_{OA}, and/or energy is transferred first from at least a portion S_{OA21} of S_{OA2} to the at least a portion S_{UA}1 of S_{UA} according to step (f) and then from the at least a portion S_{OA21} of S_{OA2} to S_{OA}.

26. The process of claim 22, wherein at least two of the columns selected from rectification column RD_{A}, reaction column RR_{A} and, if step (a2) is conducted, reaction column RR_{B} are in one column shell, and wherein columns are at least partially separated from one another by a dividing wall extending to the bottom of the columns.

27. The process of claim 22, wherein a portion of the vapour stream S_{OA} other than [symbol] , [symbol] , is separated from S_{OA}, and [symbol] is then used in step (a1) as reactant stream S_{AE1} and, if step (a2) is conducted, alternatively or additionally in step (a2) as reactant stream S_{BE1}.

28. The process of claim 27, wherein stream [symbol] is compressed after the vapour stream S_{OA} has been separated off and before it is used in step (a1) as reactant stream S_{AE1} and, if step (a2) is conducted, alternatively or additionally in step (a2) as reactant stream S_{BE1}.

29. The process of claim 22, wherein a portion of the vapour stream S_{OA1} other than S_{OA11} and S_{OA12}, S_{OA♥}, is separated from S_{OA1}, and S_{OA♥} is then used in step (a1) as reactant stream S_{AE1} and, if step (a2) is conducted, alternatively or additionally in step (a2) as reactant stream S_{BE1}.

30. The process of claim 29, wherein stream S_{OA♥} is compressed after stream S_{OA1} has been separated off and before it is used in step (a1) as reactant stream S_{AE1} and, if step (a2) is conducted, alternatively or additionally in step (a2) as reactant stream S_{BE1}.

31. The process of claim 22, wherein a portion of the vapour stream S_{OA2} other than S_{OA21}, S_{OA♦}, is separated from S_{OA2}, and S_{OA♦} is then used in step (a1) as reactant stream S_{AE1} and, if step (a2) is conducted, alternatively or additionally in step (a2) as reactant stream S_{BE1}.

32. The process of claim 31, wherein stream S_{OA♦} is compressed after the vapour stream S_{OA2} has been separated off and before it is used in step (a1) as reactant stream S_{AE1} and, if step (a2) is conducted, alternatively or additionally in step (a2) as reactant stream S_{BE1}.

33. The process of claim 22, wherein the at least a portion of stream S_{XA} is compressed after energy has been transferred thereto from the at least a portion of S_{AP} in step (g) and, if step (a2) is conducted, additionally or alternatively, the at least a portion of stream S_{XB} is compressed after energy has been transferred thereto from the at least a portion of S_{BP} in step (g).

34. The process of claim 22, wherein in step (g), S_{XA} and S_{XB} are each independently selected from S_{OA} or S_{OA1}.

35. The process of claim 34, wherein in step (g), S_{XA} and S_{XB} are each S_{OA}.

36. The process of claim 35, wherein energy is transferred from at least a portion of a stream selected from S_{OA1} or S_{OA2}, to the crude product RP_{A} and, if step (a2) is conducted, alternatively or additionally to the crude product RP_{B}.

37. The process of claim 22, wherein energy is used by at least a portion of a stream selected from S_{OA1} or S_{OA2}, in a process for preparing an alkoxide M_{C}OR″, where M_{c}OR′ is reacted with R″OH in said process to give a crude product comprising M_{C}OR″ and R′OH, with or without R″OH;
wherein R′ and R″ are two different C₁ to C₇ hydrocarbon radicals, and Mc is a metal selected from lithium, sodium, and potassium.

38. The process of claim 37, wherein, in a reactive rectification column RR_{C}, a reactant stream S_{CE1} comprising M_{c}OR′ is reacted in countercurrent with a reactant stream S_{CE2} comprising R″OH, to give a crude product RP_{C} comprising M_{C}OR″ and R′OH;
wherein a bottom product stream S_{CP} comprising M_{C}OR″ is withdrawn at the lower end of RR_{C} and a vapour stream S_{CB} comprising R′OH is withdrawn at the upper end of RR_{C};
and wherein energy is transferred from at least a portion of a stream selected from S_{OA1} or S_{OA2} to the crude product RP_{C}.

39. The process of claim 38, wherein R′=methyl or ethyl.

40. The process of claim 39, wherein at least a portion of S_{AP} is used as S_{CE1}.

41. The process of claim 39, wherein at least a portion of S_{BP} is used as S_{CE1}.
